(19) United States
(12) Patent Application Publication
(10) Pub. No.: US 2022/0187274 A1
(43) Pub. Date: Jun. 16, 2022

(54) NEMATODE TRAP PLATE AND USE THEREFOR

(71) Applicant: NATIONAL INSTITUTES FOR QUANTUM SCIENCE AND TECHNOLOGY, Chiba-shi, Chiba (JP)

(72) Inventors: Michiyo Suzuki, Chiba-shi (JP); Yuya Hattori, Chiba-shi (JP); Toshiyuki Saito, Chiba-shi (JP); Yoshinobu Harada, Chiba-shi (JP)

(21) Appl. No.: 17/605,337

(22) PCT Filed: Apr. 24, 2020
(86) PCT No.: PCT/JP2020/017674

§ 371 Date: Oct. 21, 2021

(30) Foreign Application Priority Data
Apr. 25, 2019 (JP) 2019-084499

(51) Int. Cl. G01N 33/48; G01N 33/50; G01N 33/493

(52) U.S. Cl. CPC G01N 33/48; G01N 33/493; G01N 33/5085

(57) ABSTRACT

Provided is a test plate with which a test can be carried out in a simple manner and problems that may otherwise occur along with the lapse of time hardly occur. This invention provides a nematode trap plate (1) including: a container (10); and a solid medium (11) formed in the container (10), the solid medium (11) allowing nematodes to move over its surface, and the surface of the solid medium (11) having at least one fall-in cavity (2a) for catching nematodes.

### TECHNICAL FIELD

The present invention relates to a nematode trap plate and use thereof. More specifically, the present invention relates to a nematode trap plate, a forming mold for producing the nematode trap plate, a method for producing the nematode trap plate, a container for producing the nematode trap plate, a method for analyzing a response of nematodes with use of the nematode trap plate, a method for analyzing behaviors of nematodes with use of the nematode trap plate, a nematode trap test kit including the nematode trap plate, and a method for screening cancer.

### BACKGROUND ART

Nematode Caenorhabditis elegans (it may sometimes be referred to as “C. elegans”) is a model organism for researches such as neurobiology, developmental biology, and gerontology. In 2015, an article (Non-Patent Literature 1) on a cancer screening test for humans that uses urine and the nematode C. elegans as a biosensor was published. The test according to this article is gathering attention around the world, since this test is non-invasive and has a high cancer diagnosis accuracy, reportedly a cancer diagnosis accuracy of not less than 95%.

### CITATION LIST

### Non-patent Literature

[Non-patent Literature 1]

T. Hirotsu, et al., PLOS ONE, 3, e0118699, 2015

### SUMMARY OF INVENTION

### Technical Problem

Unfortunately, however, the above-described screening test still has problems. Specifically, the above-described screening test is troublesome to be used as a simple cancer screening test. Furthermore, with that method, diagnostic accuracy cannot be sufficiently guaranteed in a single test. Thus, the above test has not yet been practically used.

For the purpose of avoiding a situation in which nematodes having once been attracted to a chemical substance area move, as time passes, to the outside of the chemical substance area, a conventional chemotaxis evaluation method involving use of nematode C. elegans carries out the following procedure, for example. That is, a liquid to be tested (it may sometimes be referred to as a “test solution”) is dripped onto one side of an agar plate, and a standard solution used as a control is dripped onto another side, which is opposite to the one side, of the agar plate. After these solutions are dried, a so-called anesthetic, such as sodium azide (NaN₃), is dripped onto each of the two sides, and then is dried for a certain duration. Consequently, a nematode having been moved to the area onto which the test solution or the standard solution has been dripped is caught (hereinafter, that may sometimes be expressed as “trapped”). This, however, takes time to dry the test solution and the anesthetic. In addition, the anesthetic diffuses over time, and this may affect the evaluation on a response to a substance to be tested and/or the like. Particularly, the latter may cause a phenomenon disadvantageous to the response evaluation, specifically, a phenomenon that once a nematode intrudes into an area onto which the anesthetic has been dripped, the nematode cannot exit therefrom irreverently to its inherent response. This leads to a problem that the accuracy of nematode response evaluation cannot be guaranteed. Moreover, the lapse of time may affect adaptation to the chemical substance and/or associative learning in the nematodes. Due to this, the nematodes may not remain within the chemical substance area into which they have once attracted, but may move to the outside of the area, although this varies depending on the concentration of the anesthetic. Furthermore, due to the above-described effects of the anesthetic on adaptation over time, and/or the like, the result may vary each time the test is carried out, disadvantageously. In order to deal with these disadvantages, time-lapse photographing may be carried out to capture the behaviors of the nematodes. This, however, requires a device for carrying out time-lapse photography, and consequently the procedure for the test becomes complex.

There is further another problem. That is, according to the conventional chemotaxis evaluation method on the nematode C. elegans, after a certain duration has elapsed since start of the test, the number of nematodes within the area of the substance to be tested or the like and the number of nematodes within the control area are counted through visual observation or observation on an image of the entire agar plate having been captured. However, since the range to be observed or the range to be imaged is wide relative to the size of each nematode, it takes labor to count the numbers.

In light of the above problems, the present invention was made. An object of an aspect of the present invention is to provide a nematode trap plate with which (a) a test can be carried out in a simple manner, (b) problems that may otherwise occur along with the lapse of time hardly occur, and (c) a response would not be evaluated erroneously due to the effect of an anesthetic.

### Solution to Problem

In order to attain the object, a nematode trap plate in accordance with an aspect of the present invention includes: a container; and a solid phase formed in the container, the solid medium allowing nematodes to move over a surface of the solid phase, the surface of the solid phase having at least one recess for catching one or some of the nematodes.

In the nematode trap plate in accordance with the aspect of the present invention, the container should have a bottom surface having a transmittance of 70% or more for light having a wavelength of 360 nm to 1500 nm.

A forming mold in accordance with an aspect of the present invention is a forming mold for producing the above-described nematode trap plate, the forming mold including: a main body; and at least one protrusion provided in the main body, said at least one protrusion corresponding to said at least one recess, the forming mold being fixable to the container.

In the forming mold in accordance with the aspect of the present invention, said at least one protrusion is preferably detachably attachable to the main body.

A container in accordance with an aspect of the present invention is a container for producing the above-described nematode trap plate, the container including: a hole provided at a part of a bottom surface of the container at which part said at least one recess is to be formed, the hole being designed to receive a tubular member that is to be inserted into the hole to form said at least one recess; and a space below at least the part of the bottom surface at which part the hole is provided.

In an aspect of the present invention, a method for producing a nematode trap plate is a method for producing the above-described nematode trap plate, the method including the step of: forming said at least one recess in the solid phase, wherein, in the step of forming said at least one recess, said at least one recess is formed by (a) forming the solid phase with the above-described forming mold being fixed to the container or (b) forming the solid phase on the container, followed by hollowing out a part of the solid phase.

A method in accordance with another aspect of the present invention for producing a nematode trap plate is a method for producing the above-described nematode trap plate, the method including the step of: forming said at least one recess in the solid phase, wherein, in the step of forming said at least one recess, said at least one recess is formed by (a) inserting, into the hole of the container recited in claim 5, a tubular member that has a bottom at a lower end of the tubular member and that is opened at an upper end of the tubular member, so that the tubular member is fixed at the hole, (b) forming the solid phase so that the surface of the solid phase is lower in height than the upper end of the tubular member, and (c) after forming the solid phase, pushing down the tubular member until the upper end of the tubular member coincides in height with the surface of the solid phase.

In order to attain the object, a method in accordance with an aspect of the present invention for evaluating a response of nematodes is a method for evaluating a response of nematodes to a test subject, the method including the steps of: preparing a test plate including the above-described nematode trap plate in which a test subject has been supplied into said at least one recess or to an area around said at least one recess; supplying nematodes to a certain position on the surface of the solid phase; and after a certain duration has elapsed, obtaining the number of nematodes caught in said at least one recess or a factor that correlates with the number of nematodes caught in said at least one recess, wherein said at least one recess is filled with a liquid.

In an aspect of the present invention, the method for evaluating the response of the nematodes, the test subject encompasses a body fluid of mammal.

In an aspect of the present invention, the method for evaluating the response of the nematodes, the test subject encompasses urine of human, canine, feline, monkey, mouse, rat, or marmot.

In order to attain the object, a method in accordance with an aspect of the present invention for evaluating a response of nematodes is a method for evaluating a response of nematodes to a temperature, the method including the steps of: adjusting a temperature inside said at least one recess or a temperature of an area around said at least one recess in the above-described nematode trap plate so that the temperature is at a desired value; supplying nematodes to a certain position on the surface of the solid phase; and after a certain duration has elapsed, obtaining the number of nematodes caught in said at least one recess or a factor that correlates with the number of nematodes caught in said at least one recess, wherein said at least one recess is filled with a liquid.

In an aspect of the present invention, the method for evaluating the response of the nematodes, after the certain duration has elapsed, an image of said at least one recess is captured and image processing is carried out on the image thus captured so as to obtain the number of nematodes caught in said at least one recess or the factor that correlates with the number of nematodes caught in said at least one recess.

In an aspect of the present invention, the method for evaluating the response of the nematodes, the nematodes to be used are nematodes having a fluorescent probe incorporated therein, and the number of nematodes caught in said at least one recess is calculated from a total of fluorescence intensities of the nematodes caught in said at least one recess.

In order to attain the object, a method in accordance with an aspect of the present invention for evaluating behaviors of nematodes includes the steps of: supplying nematodes to a certain position on the surface of the solid phase of the above-described nematode trap plate; and after a certain duration has elapsed, obtaining the number of nematodes caught in said at least one recess or a factor that correlates with the number of nematodes caught in said at least one recess, wherein said at least one recess is filled with a liquid.

In order to attain the object, a nematode trap test kit in accordance with an aspect of the present invention includes the above-described nematode trap plate; and a cover for maintaining an environment on the solid phase so as to be fixed, the cover having a transmittance of not less than 70% for light having a wavelength of 360 nm to 1500 nm.

In an aspect of the present invention, the nematode trap test kit further includes nematodes to be used in a test.

In order to attain the object, a method in accordance with an aspect of the present invention for screening cancer includes the steps of: preparing a test plate including the above-described nematode trap plate in which urine obtained from a testee has been supplied into said at least one recess or to an area around said at least one recess; supplying nematodes to a certain position on the surface of the solid phase; and after a certain duration has elapsed, obtaining the number of nematodes caught in said at least one recess or a factor that correlates with the number of nematodes caught in said at least one recess, wherein said at least one recess is filled with a liquid, and a possibility that the testee has cancer is determined based on the number of nematodes caught in said at least one recess.

In an aspect of the present invention, the method for screening cancer is configured such that the testee is human.

In an aspect of the present invention, the method for screening cancer is configured such that the testee is canine or feline.

In an aspect of the present invention, the method for screening cancer is configured such that the testee is monkey, mouse, rat, or marmot.

### Advantageous Effects of Invention

In a response evaluation test employing a nematode trap plate in accordance with an aspect of the present invention, problems that may otherwise occur along with the lapse of time hardly occur, and therefore it is possible to evaluate the response in a simpler manner with high accuracy.

### BRIEF DESCRIPTION OF DRAWINGS

FIG. 1 is a view schematically illustrating an aspect of a nematode trap plate and a cover. (a) of FIG. 1 is a top view of the nematode trap plate, (b) of FIG. 1 is a cross-sectional view of the nematode trap plate, and (c) of FIG. 1 is a cross-sectional view of the nematode trap plate covered with the cover.

FIG. 2 is a view schematically illustrating another aspect of the nematode trap plate. (a) of FIG. 2 is a top view of the nematode trap plate, (b) of FIG. 2 is a cross-sectional view of the nematode trap plate shown in (a) of FIG. 2, (c) of FIG. 2 is a top view of another nematode trap plate, (d) of FIG. 2 is a cross-sectional view of the nematode trap plate shown in (c) of FIG. 2, (e) of FIG. 2 is a top view of further another nematode trap plate, and (f) of FIG. 2 is a cross-sectional view of the nematode trap plate shown in (e) of FIG. 2.

FIG. 3 is a perspective view of some aspects of appearances of hollowing-out tools.

FIG. 4 is a view illustrating some aspects of appearances of forming molds having different configurations.

(a) of FIG. 5 is a top view of a fit-in type forming mold used to produce a nematode trap plate in accordance with an embodiment, and (b) of FIG. 5 is a perspective view of a cross section of the forming mold.

(a) of FIG. 6 is a top view of a container used to produce a nematode trap plate in accordance with an embodiment, and (b) of FIG. 6 is a cross-sectional view of the container. (c) to (e) of FIG. 6 are views illustrating a process of producing the nematode trap plate with use of the container.

FIG. 7 is a view illustrating one example of a still image of C. elegans having been caught in a fall-in cavity 2a filled with a diluted solution of diacetyl in Example 1.

FIG. 8 is a view illustrating one example of a still image of C. elegans having been caught in a control fall-in cavity 2b filled with a buffer solution in Example 1.

(a) to (c) of FIG. 9 are views illustrating the results of three times of tests carried out in Example 1 for evaluating a response of C. elegans to diacetyl.

FIG. 10 is a view illustrating mean values of the results of the tests carried out in Example 1 for evaluating the response of C. elegans to diacetyl.

FIG. 11 is a view illustrating one example of a still image of an entire assay plate captured in Comparative Example 1.

(a) to (c) of FIG. 12 are views illustrating the results of three times of tests carried out in Comparative Example 1 for evaluating a response of C. elegans to diacetyl.

FIG. 13 is a view illustrating mean values of the results of the tests carried out in Comparative Example 1 for evaluating the response of C. elegans to diacetyl.

(a) to (c) of FIG. 14 are views illustrating the results of three times of tests carried out in Example 2 for evaluating a response of C. elegans to diacetyl.

FIG. 15 is a view illustrating mean values of the results of the tests carried out in Example 2 for evaluating the response of C. elegans to diacetyl.

FIG. 16 is a view illustrating the results of tests carried out in Example 3 for evaluating a response of C. elegans to benzaldehyde.

FIG. 17 is a view illustrating the results of tests carried out in Example 4 for evaluating a response of C. elegans to the urine of a patient with a prostate cancer.

FIG. 18 is a view illustrating the results of tests carried out in Example 5 for evaluating a response of C. elegans to the urine of a healthy individual having never developed cancer.

FIG. 19 is a view illustrating the results of tests carried out in Example 6 for evaluating a response of C. elegans to the urine of a male canine having never developed cancer.

FIG. 20 is a view illustrating the results of tests carried out in Example 7 for evaluating a response of C. elegans to the urine of a female canine having never developed cancer.

FIG. 21 is a view illustrating the results of tests carried out in Example 8 for evaluating a response of C. elegans to the urine of (a plurality of) canines with cancer.

FIG. 22 is a view illustrating the results of tests carried out in Example 9 for evaluating a response of C. elegans to the urine of (a plurality of) canines having never developed cancer.

FIG. 23 is a view illustrating the results of tests carried out in Example 10 for evaluating a response of C. elegans to the urine of (a plurality of) felines with cancer.

FIG. 24 is a view illustrating the results of tests carried out in Example 11 for evaluating a response of C. elegans to the urine of (a plurality of) felines having never developed cancer.

FIG. 25 is a view illustrating the results of tests carried out in Example 12 for evaluating a response of C. elegans to the urine of (a plurality of) rats with mammary gland cancer.

FIG. 26 is a view illustrating the results of tests carried out in Example 13 for evaluating a response of C. elegans to the urine of (a plurality of) rats having never developed cancer.

FIG. 27 is a view illustrating the results of tests carried out in Example 14 for evaluating a response of C. elegans to sodium chloride.

FIG. 28 is a view illustrating the results of tests carried out in Example 15 for evaluating a response of soil nematodes other than C. elegans to vanillin.

FIG. 29 is view illustrating one example of a processing screen of an image analyzing program used in Example 16 to automatically count the number of nematode individuals.

FIG. 30 is view illustrating the result obtained as a result of automatic counting of the number of nematode individuals carried out in Example 16.

### DESCRIPTION OF EMBODIMENTS

An embodiment of the present invention will be described below with reference to the drawings as needed.

[Nematode Trap Plate]

(Nematode)

The term “nematode” herein means both an organism belonging to phylum Nematoda (hereinafter, simply referred to as “Nematoda”) and an organism belonging to phylum Nematomorpha (hereinafter, simply referred to as “Nematomorpha”) according to the biological classifications. The nematode is not limited to any specific one, and may be any organism that is included in the above-described classifications, that is terrestrial or semiterrestrial, and that is able to move over a solid phase.

Examples of Nematoda encompass various kinds of nematodes such as nonparasitic nematodes (or free-living nematodes), plant-parasitic nematodes, entomogenous nematodes, phoretic nematodes of insects (and the like), and parasitic nematodes of mammals (and the like).

Examples of the nonparasitic nematode encompass Caenorhabditis elegans (hereinafter, sometimes referred to as “C. elegans”), Aphelenchus avenae, Caenorhabditis angaria, Caenorhabditis brenneri, Caenorhabditis briggsae, Caenorhabditis japonica, Caenorhabditis remanei, and Pristionchus pacificus.

Examples of the plant-parasitic nematode encompass Meloidogyne incognita, Meloidogyne arenaria, Meloidogyne javanica, Meloidogyne hapla, Heterodera glycines, Globodera rostochiensis, Globodera pallida, Pratylenchus penetrans, Pratylenchus coffeae, Pratylenchus vulnus, Ditylenchus dipsaci, Aphelenchoides besseyi, Anguina tritici, Ditylenchus destractor, Aphelenchoides ritzemabosi, Longidorus spp., Xiphinema index, Aphelenchoides fragariae, Pratylenchus brachyurus, Caenorhabditis inopinata, and Xiphinema brevicolle.

Examples of the entomogenous nematode encompass Sphaerularia bombi, Sphaerularia vespae, Hexamermis zuimyshi, Steinernema carpocapsae, Steinernema kushidai, Iotonchium ungulatum, Iotonchium californicum, Iotonchium cateniforme, Iotonchium laccariae, Iotonchium russulae, Caenorhabditis auriculariae, and Bursaphelenchus tadamiensis.

Examples of the phoretic nematodes of insects (and the like) encompass Caenorhabditis japonica, Pristionchus pacificus, Bursaphelenchus xylophilus, Bursaphelenchus conicaudatus, Bursaphelenchus luxuriosae, Teratorhabditis synpapillata, Caenorhabditis briggsae, and Caenorhabditis remanei. Among these, Caenorhabditis japonica, Pristionchus pacificus, Caenorhabditis briggsae, and Caenorhabditis remanei are dealt with as similar organisms to the free-living nematodes (nonparasitic nematodes) in a laboratory.

Examples of the parasitic nematode of mammals (and the like) encompass threadworms, filariae, roundworms, Anisakis, whipworms, hookworms, Gnathostomata, and trichinae.

Examples of the threadworm are shown below according to the classifications of main hosts: threadworms parasitic on animals of the order Anura of the class Amphibia (Strongyloides pereira (hereinafter, “Strongyloides”, which is the generic name of the threadworm, may sometimes be abbreviated simply as “S.”), S. carinii, S. amphibiophilus, S. bufonis, S. physali, S. spiralis, S. prokopici, S. mascomai, etc.); threadworms parasitic on animals of the order Lacertilia of the class Reptilia (S. cruzi, S. darevskyi, S. ophiusensis, etc.); threadworms parasitic on animals of the order Ophidia of the class Reptilia (S. ophidiae, S. mirzai, S. gulae, S. serpentis, etc.); threadworms parasitic on animals of the order Ciconiiformes of the class Ayes (S. cubaensis, S. ardeae, S. herodiae, etc.); threadworms parasitic on animals of the order Galliformes of the class Ayes (S. avium, S. oswaldoi, S. pavonis, etc.); threadworms parasitic on animals of the order Anseriformes of the class Ayes (S. minimum, etc.); threadworms parasitic on animals of the order Charadriiformes of the class Ayes (S. turkmenica, etc.); threadworms parasitic on animals of the order Passeriformes of the class Ayes (S. quiscali Barus, etc.); threadworms parasitic on animals of the order Marsupialia of the class Mammalia (S. thylacis, etc.); threadworms parasitic on animals of the order Insectivora of the class Mammalia (S. akbari, S. rostombekowi, etc.); threadworms parasitic on animals of the order Primates of the class Mammalia (S. stercoralis, S. fuelleborni, S. fuelleborni kelly, S. cebus, etc.); threadworms parasitic on animals of the order Xenarthra of the class Mammalia (S. dasypodis, S. shastensis, etc.); threadworms parasitic on animals of the Pholidota of the class Mammalia (S. leiperi, etc.); threadworms parasitic on animals of the order Rodentia of the class Mammalia (S. chapini, S. ratti, S. myopotami, S. venezuelensis, S. agoutii, S. robustus, S. sigmodontis, etc.); threadworms parasitic on animals of the order Carnivora of the class Mammalia (S. nasua, S. felis, S. mustelorum, S. erschowi, S. planiceps, S. puttori, S. martis, S. vulpis, S. tumefasciens, S. lutrae, S. procyonis, etc.); threadworms parasitic on animals of the order Proboscidea of the class Mammalia (S. elephantis, etc.); threadworms parasitic on animals of the order Perissodactyla of the class Mammalia (S. westeri, etc.); and threadworms parasitic on animals of the order Artiodactyla of the class Mammalia (S. papillosus, S. ransomi, etc.). Among these, S. stercoralis, S. fuelleborni, and S. fuelleborni kellyi are so-called human threadworms, which are parasitic to humans. In addition to them, S. procyonis, which is a raccoon threadworm, and S. ransomi, which is a pig threadworm, may sometimes be parasitic on humans.

Examples of the filaria encompass Dirofilaria immitis, Wuchereria bancrofti, Loa loa, Onchocerca volvulus, and Brugia malayi.

Examples of the roundworm encompass a human roundworm (Ascaris lumbricoides), a pig roundworm (Ascaris suum), Lagochilascaris minor, a cattle roundworm (Neoascaris vitulorum), a horse roundworm (Parascaris equorum), a raccoon roundworm (Baylisascaris procyonis), a canine roundworm (Toxocara canis), and a feline roundworm (Toxocara cati).

Examples of Anisakis encompass so-called Anisakis type I, such as Anisakis pegreffii, Anisakis simplex sensu stricto, and Anisakis simplex C, Anisakis type II (Anisakis physeteris), and Psudoterranova decipiens.

Examples of the whipworm encompass a human whipworm (Trichuris trichiura).

Examples of the hookworm encompass a Dubini hookworm (Ancylostoma duodenale), an American hookworm (Necator americanus), and a canine hookworm (Ancylostoma caninum).

Examples of Gnathostomata encompass Gnathostoma nipponicum, Gnathostoma spinigerum, Gnathostoma hispidum, and Gnathostoma doloresi.

Examples of the trichina encompass Trichinella britovi, Trichinella spiralis, Trichinella nativa, Trichinella nelsoni, and Trichinella pseudospiralis.

Examples of the parasitic nematodes of mammals (and the like) encompass capillary nematodes (also referred to as Capillaria), such as Capillaria philippinensis and Capillaria aerophila, and Thelazia callipaeda.

Examples of Nematomorpha encompass horsehair worms. Examples of Gordioidea encompass Gordius robustus, Gordius ogatai, Pseudogordius tanganykae, and Chordodes japonensis.

(Nematode trap plate)

A nematode trap plate (hereinafter, it may sometimes simply be referred to as a “plate”) in accordance with an embodiment of the present invention is a plate including a container having a solid phase formed therein, the solid phase having a surface provided with at least one recess (a fall-in cavity, which will be describer later). The nematode trap plate in accordance with the embodiment can also be referred to as a pond assay for sensory system (PASS) plate.

The solid phase herein means a solid-phase layer formed in the container. The solid phase is not limited to any particular one, provided that it allows nematodes to mover over its surface. Specifically, as the solid phase, a layer containing moisture and having a wet surface due to the moisture is assumed. Non-limiting examples of the solid-phase layer encompass a gel made of agar, agarose, gelatin, konjac, or the like and a gel prepared by adding, to a liquid, a gelling agent or a thickening stabilizer such as pectin, guar gum, carrageenan, or xanthan gum. In order to allow nematodes to move over a medium for organisms, it is preferable to use a solid medium obtained through solidification or gelation with use of agar or the like. Meanwhile, in order not to inhibit the biological characteristics of the nematode, i.e., in order to achieve biocompatibility, it is preferable to use a solid medium obtained through solidification or gelation with use of a naturally-derived material such as agar. Furthermore, it is preferable to use a solid medium that is tasteless and odorless, in order to prevent a situation in which the taste or odor of the solid medium affects the test involving use of the nematode trap plate. To the solid medium, a sulfur source, phosphate, and a small amount of mineral can be added. For example, magnesium sulfate (MgSO₄), potassium dihydrogenphosphate (KH₂PO₄), dipotassium hydrogenphosphate (K₂HPO₄), calcium chloride (CaCl₂), and/or the like may be added to the solid medium. The solid phase can be, for example, a medium obtained through solidification or gelation of a medium for organisms. For convenience of explanation, the description below deals with an example case using, as the solid phase, a solid medium obtained by solidifying or gelating a medium for organisms with use of agar or the like. However, this is not limitative.

In order to prevent the nematode from intruding into the inside of the solid medium, the solid medium has a durometer hardness of preferably not less than 5, more preferably not less than 8, even more preferably not less than 15, as measured by a rubber durometer in accordance with the method in compliance with the standards of the physical testing method for molded products of thermosetting polyurethane elastomers defined in JIS K 7312 (type C). Setting the durometer hardness so as to be not less than 5 makes it possible to prevent a phenomenon that the nematode swimming in a liquid phase, such as in a liquid drop on the surface of the solid medium and a liquid in the recess, that is in contact with the solid medium intrudes into the inside of the solid medium. Setting the durometer hardness so as to be not less than 8 makes it possible to prevent a phenomenon that the nematode, which is used in a stress (e.g., starvation) free condition, intrudes into the inside of the solid medium.

In order that the state of the solid medium is maintained so as to be fixed during the test or during a preservation period, the solid medium is preferably a solid medium from which moisture is hardly evaporated or a solid medium that is difficult to be dried. Examples of such a solid medium encompass konjac. Meanwhile, in a case where a solid medium that is relatively easy to dried, such as agar or gelatin, is to be used, the plate may be covered and sealed with tape or may be preserved in a sealing case, for the purpose of preventing drying.

Note that the solid phase only needs to be formed as a layer having a surface over which the nematodes can move, and may have another layer (lower layer) provided below that layer. For example, usable as the surface layer can be a layer that can suitably facilitate movement of the nematode and diffusion of a subject to be tested (test subject, which will be described later), whereas usable as the lower layer can be a layer that can suitably prevent intrusion of the nematode into its inside and diffusion of the test subject. Note that, in a case of employing a solid phase made of two layers, the layers to be included are not limited to the layers having the above-described characteristics. Alternatively, a solid phase made of a three or more layers may be employed.

As will be described in details later, the plate has a recess (fall-in cavity) formed therein, and a liquid is poured into the recess when the plate is to be used. After the recess is formed in the solid medium, a recess protection wall including, as a basic material, a material that does not absorb a liquid or a material that does not allow a liquid to pass therethrough may be disposed in close contact with the inner wall of the recess. Examples of such a material encompass plastic, glass, and a silicone resin such as polydimethylsiloxane (hereinafter, it may sometimes be referred to as “PDMS”). With this, it is possible to prevent a phenomenon that a liquid poured into the recess or a component(s) included in the liquid penetrates into the solid medium. In addition, it is also possible to prevent a phenomenon that a liquid contained in the solid medium penetrates into the recess. Note that the “liquid contained in the solid medium” herein refers to a liquid that is a constituent of the solid medium and that is used to form the solid medium.

The container in which the solid medium is to be formed is not particularly limited in terms of size, shape, and material, and may be a commercially-available container, such as a plastic dish.

The material of the container in which the solid medium is to be formed is not limited to any particular one, and may be plastic, glass, PDMS, or the like. Preferably, the container is designed to allow microscopic observation through a bottom surface of the container. That is, it is preferable that the bottom surface of the container have a visible light/ultraviolet light transmittance enough for microscopic observation. Specifically, the bottom surface of the container has a transmittance of preferably not less than 70%, more preferably not less than 80%, even more preferably 90% for light having a wavelength of 360 nm to 860 nm. In addition, the transmittance is preferably not less than 70%, more preferably not less than 80%, even more preferably 90% for light having a wavelength of 860 nm to 1500 nm. Furthermore, the bottom surface of the container may be the one including, as a basic material, a material that does not emit autofluorescence or emits less autofluorescence. The container including such a basic material is suitably applicable to fluorescence observation. For example, in a case where plastic is used as the basic material, plastic having excellent light transmittance and emitting less autofluorescence, such as polyester such as polyethylene terephthalate (PET), polymethyl methacrylate (PMMA), polycarbonate (PC), a cycloolefin polymer (COP), or an epoxy resin, may be adopted.

The shape of the container, typically, the inner shape of the container can be circular, rectangular, or square, for example. Note that, herein, the shape of the container means the shape of the container as viewed from above.

In addition, the bottom surface of the container may be provided with a guide indicating the position where the later-described fall-in cavity is to be formed or a guide indicating the position to which the nematode is to be supplied.

The size of the container can be arbitrarily selected in accordance with the size of the nematode to be used. For example, in a case where C. elegans is used as the nematode, a container having a bottom surface with a maximum diameter (inner diameter) ranging from 3 cm to 25 cm can suitably be used. Here, in a case of a circular container, the maximum diameter means the diameter of the container. In a case of a rectangular container, the maximum diameter means the length of a long side of the container. In a case of a square container, the maximum diameter means the length of one side of the container. Specific examples of a suitable container encompass a circular dish (e.g., a plastic dish) having a bottom surface with an inner diameter of 5.2 cm and a polygon dish (e.g., a plastic dish) having a bottom surface shaped in a 6 cm-square. In a case where such a container is used, the thickness of a solid medium therein can be not more than approximately 2 cm.

In a case where an integrated microscope (including an integrated fluorescence microscope) having an automatic imaging function is used to count the number of nematodes through image analysis (described later) or in a case where an analysis system that is constituted by a stereo microscope (including a fluorescence stereo microscope), an image capturing device, and an image processing device and that is specialized in number counting through image analysis is constructed, it is preferable to select the shape and size of the container in accordance with the sample stage of the device.

(Fall-In Cavity)

The nematode trap plate in accordance with the embodiment includes the solid medium having the surface provided with at least one recess for catching nematodes. The recess is also called a “fall-in cavity”.

Herein, “catching” is synonymous with “trapping”, and both mean keeping, inside a certain area, nematodes having intruded into the certain area so as to prevent the nematodes from exiting from the certain area. The “catching” or “trapping” only needs to keep the nematodes within the certain area for a certain duration, that is, to keep the nematodes within the certain area and prevent the nematodes from exiting from the certain area for a certain duration required in specific aspects to carry out the test and then to count the number of nematodes. For example, there may be a case where a certain duration from starting of the test to counting of the number of nematodes having been caught or collecting these nematodes is less than an hour. In such a case, the “catching” or “trapping” only needs to prevent, for an hour, the nematodes from exiting from the area. In order to impart carry out the test and the number counting with the degree of freedom, it is preferable to prevent the nematodes from exiting from the area for a long duration. For example, such a duration is preferably not shorter than two hours, more preferably not shorter than 12 hours.

The fall-in cavity is formed so as to extend from the surface of the solid medium toward the bottom of the solid medium. The fall-in cavity may or may not reach the bottom surface of the solid medium. For example, the fall-in cavity may have a depth almost equal to the thickness of the solid medium.

The fall-in cavity may be a tube that is detachably embedded in advance in the solid medium and that is opened upward. In this case, a raised-bottom container may be employed, and a tube may be fitted into a hole having been formed in the bottom surface of the container. In the case where the fall-in cavity is produced by fitting the tube into the raised-bottom container, the depth of the fall-in cavity is typically equal to or greater than the thickness of the solid medium, although it varies depending on the thickness of the bottom of the tube to be embedded and the depth of the container of the plate.

The fall-in cavity may be configured such that its bottom is openable. The configuration in which the fall-in cavity has the openable bottom enables the followings. That is, when the test is to be carried out, the bottom is closed so that the fall-in cavity is used as a fall-in cavity. Meanwhile, after the test is ended, the bottom is opened so that the nematodes having been caught therein can be collected through the bottom.

The number of fall-in cavities and the shape(s) and position(s) of the fall-in cavity(ies) are not particularly limited, and may be appropriately set in accordance with the kind of nematodes to be used and the number thereof, the kind of test subject and the number thereof, the purpose of the test, and/or the like. For example, the number of fall-in cavities can be one, two, three, four, five, six or more. The shape of the fall-in cavity may be circular, rectangular, square, elliptical, or polygonal, for example. Herein, the shape of the fall-in cavity means the shape of the fall-in cavity as viewed from above. The fall-in cavity is typically provided at a position in the vicinity of the outer periphery of the plate. However, this is not limitative. The fall-in cavity may be formed such that a part of the outer periphery of the plate constitutes a part of the fall-in cavity. For example, the fall-in cavity may be an arch-shaped fall-in cavity formed by cutting a part of the outer periphery of a solid medium in a circular plate, a linear fall-in cavity formed by cutting a side of a solid medium of a polygon plate, or a triangular fall-in cavity formed by cutting an edge of a solid medium of a polygon plate. For another example, the fall-in cavity may be a fall-in cavity having a moat-like structure in which a solid medium is positioned inward of the fall-in cavity. In a case of employing the fall-in cavity having a moat-like structure in which the solid medium is positioned inward of the fall-in cavity, even if a test subject is insoluble in a liquid, for example, the test subject can be placed on the solid medium positioned inward of the fall-in cavity, so that the test subject can be disposed inward of the fall-in cavity. Alternatively, in a case of employing the fall-in cavity having a moat-like structure in which the solid medium is positioned inward of the fall-in cavity, a solid medium containing the test subject can be placed at a position inward of the fall-in cavity, so that the test subject can be disposed inward of the fall-in cavity. Further alternatively, in a case of employing the fall-in cavity having a moat-like structure in which the solid medium is positioned inward of the fall-in cavity, a hole may be formed in the solid medium positioned inward of the fall-in cavity, and a test subject may be supplied into the hole. Still alternatively, another solid medium may additionally be provided at a position inside the hole, so that a double-moat-like structure constituted by fall-in cavities can be provided. Yet alternatively, in the vicinity of the fall-in cavity, a test subject supplying hole that is not the fall-in cavity may be formed.

In a case where a fall-in cavity is formed to have a bottom surface of a greater area in order to achieve a greater volume, observation of the nematodes having been caught is facilitated. Meanwhile, for a test involving use of a solid medium having a concentration gradient of a test subject formed therein, a fall-in cavity with a greater depth and a smaller bottom area may be formed.

(Cover)

The nematode trap plate may further include a cover that is separated from the nematode trap plate and that can be used in combination with the plate during the test. The cover is used to maintain the state inside the plate so as to be fixed during the test. Covering the plate with the cover makes it possible to suppress or reduce drying of the solid medium, to suppress or reduce evaporation of the liquid filled in the fall-in cavity, and/or to maintain the humidity in the plate, for example, thereby making it possible to maintain the test environment so as to be fixed. In addition, in an olfaction test, covering the plate with the cover makes it possible to suppress or reduce diffusion of an odorant and to prevent contamination of an odorant coming from the outside of the plate, for example, thereby making it possible to maintain the test environment so as to be fixed. Furthermore, covering the plate with the cover makes it possible to prevent the nematodes from escaping from the plate. In order to further enhance the sealing, after the plate is covered with the cover, the plate may be sealed by applying hydrophobic tape and/or the like to cover the edge of the cover and fixing the tape to the outer periphery of the plate.

The cover is preferably designed to allow microscopic observation even on the plate covered with the cover. That is, preferably, the cover has a visible light/ultraviolet light transmittance enough for microscopic observation. Specifically, the cover has a transmittance of preferably not less than 70%, more preferably not less than 80%, even more preferably not less than 90% for light having a wavelength of 360 nm to 860 nm. In addition, the transmittance is preferably not less than 70%, more preferably not less than 80%, even more preferably not less than 90% for light having a wavelength of 860 nm to 1500 nm. Furthermore, the cover may be the one including, as a basic material, a material that does not emit autofluorescence or emits less autofluorescence. The cover including such a basic material is suitably applicable to fluorescence observation.

(Specific Examples of Nematode Trap Plate)

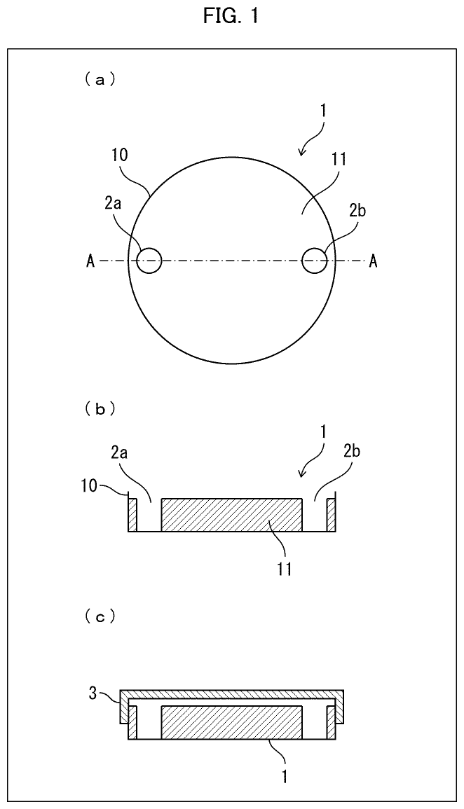

FIG. 1 illustrates one example of a plate including, as a container, a commercially-available circular plastic dish. (a) of FIG. 1 is a top view of a plate 1, and (b) of FIG. 1 is a cross-sectional view of the plate 1 taken along the broken line A-A in (a) of FIG. 1. (c) of FIG. 1 is a cross-sectional view of the plate 1 covered with a cover 3. The plate 1 is prepared by preparing a solid medium 11 through solidification or gelation of a liquid medium poured into a commercially-available circular plastic dish 10 and by forming two fall-in cavities 2a and 2b therein. The two fall-in cavities 2a and 2b are provided at positions in the vicinity of the outer periphery of the solid medium 11 and that are symmetric to each other with respect to the center of the circle. As shown in (b) of FIG. 1, in the plate 1, the fall-in cavities 2a and 2b reach the bottom of the solid medium.

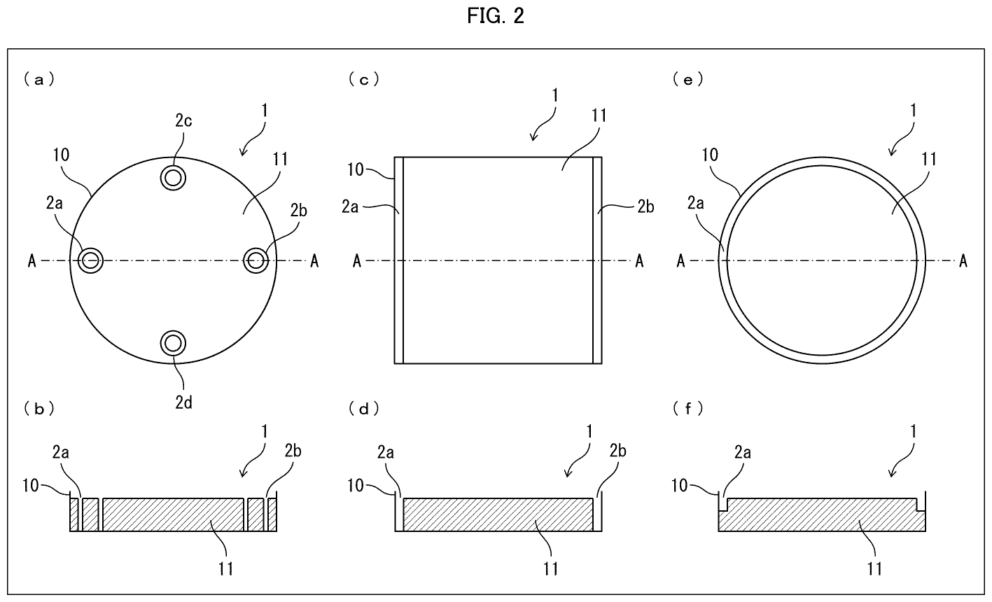

FIG. 2 illustrates a plate in accordance with another aspect. (a) of FIG. 2 is a top view of another aspect of the plate 1, and (b) of FIG. 2 is a cross-sectional view taken along the broken line A-A in (a) of FIG. 2. (c) of FIG. 2 is a top view of further another aspect of the plate 1, and (d) of FIG. 2 is a cross-sectional view taken along the broken line A-A in (c) of FIG. 2. (e) of FIG. 2 is a top view of still further another aspect of the plate 1, and (f) of FIG. 2 is a cross-sectional view taken along the broken line A-A in (f) of FIG. 2. In the example shown in (a) of FIG. 2, four fall-in cavities 2a to 2d are provided. Also at a position inward of the fall-in cavities 2a to 2d, a solid medium 11 resides. Consequently, the fall-in cavities 2a to 2d constitute a moat-like structure. In the example shown in (c) of FIG. 2, a commercially-available polygonal plastic dish is used as the container, and fall-in cavities 2a and 2b are provided as if the fall-in cavities 2a and 2b respectively cut ends of a solid medium 11. Thus, parts of the outer periphery of the solid medium 11 respectively constitute parts of the fall-in cavities 2a and 2b. In the example shown in (e) of FIG. 2, a fall-in cavity 2a is provided as if the fall-in cavity 2a cuts the entire periphery of a solid medium 11. Thus, the outer periphery of an upper part (i.e., a surface part) of the solid medium 11 constitutes the inner wall of the fall-in cavity 2a.

(Method for Forming Fall-In Cavity)

A method for forming the fall-in cavity is not limited to any particular one, provided that it can form a fall-in cavity of a desired shape. The fall-in cavity may be formed by any method. Examples of a simple method for forming the fall-in cavity encompass (i) a method of forming a fall-in cavity by forming a solid medium in a container, followed by hollowing out a part of the solid medium (first forming method), (ii) a method for forming a fall-in cavity by attaching and fixing a forming mold having a protrusion to a container in which a solid medium is not formed yet, forming a solid medium in the container to which the forming mold is fixed, and then removing the forming mold therefrom (second forming method), and (iii) a method for forming a fall-in cavity by forming a solid medium in a container in which a tubular container, which is to be turned into the fall-in cavity, is placed in advance (third forming method).

(First Forming Method)

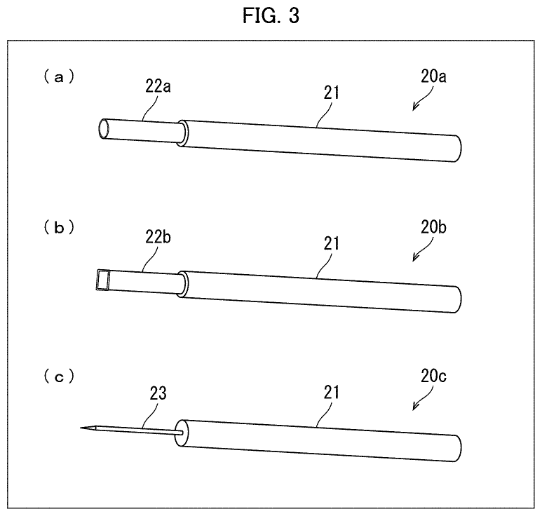

First, the following will explain the method for forming a fall-in cavity by forming a solid medium, followed by hollowing out a part of the solid medium. According to this method, first, a liquid medium is poured into a container, and then the liquid medium is solidified or gelated therein. Subsequently, in a case where a circular fall-in cavity is to be formed, a tubular (cylindrical) tool such as a straw is pierced into the medium so as to hollow out a part of the medium which part is inside the tubular tool. Consequently, a fall-in cavity is formed. The tubular tool to be used for the hollowing-out may be selected in accordance with the shape of a fall-in cavity to be formed, or may be produced by a user. FIG. 3 illustrates examples of a hollowing-out tool used to form a fall-in cavity. (a) of FIG. 3 is a perspective view of an appearance of a hollowing-out tool 20a used to form a circular fall-in cavity. (b) of FIG. 3 is a perspective view of an appearance of a hollowing-out tool 20b used to form a rectangular fall-in cavity. The hollowing-out tool 20a shown in (a) of FIG. 3 includes a hollow tubular part 22a having a circular cross-section taken along an orthogonal direction to its longitudinal direction and a grip 21 provided to the hollow tubular part 22a. The hollowing-out tool 20b shown in (b) of FIG. 3 includes a hollow tubular part 22b having a rectangular cross-section taken along an orthogonal direction to its longitudinal direction and a grip 21 provided to the hollow tubular part 22b.

The method for hollowing out a part of the solid medium is not limited to the method involving use of the tubular tool. Alternatively, this can be carried out by a method of craving, with use of a needle with a sharp point, the contour of a fall-in cavity to be formed and then hollowing out a part inside the contour. With this, it is possible to hollow out a desired shape part of the solid medium. (c) of FIG. 3 illustrates an example of a hollowing-out tool suitable for this method. The hollowing-out tool 20c shown in (c) of FIG. 3 includes a needle 23 with a sharp point and a grip 21 provided to the needle 23.

In a case where a fall-in cavity is formed by hollowing out a part of the solid medium, it is preferable that the fall-in cavity reach the bottom surface of the solid medium, that is, the bottom surface of the container coincide with the bottom surface of the fall-in cavity, in order that the fall-in cavity has a bottom surface of a certain uniform shape.

(Second Forming Method)

Next, the following will explain the method for forming a fall-in cavity with use of a forming mold (also referred to as a mold). First, the forming mold will be explained.

The forming mold in accordance with the embodiment is a tool that can be fixed to an upper surface of the container and that has a main body part typically having a plate shape and having one surface provided with at least one protrusion. The forming mold is not limited to the one having the main body part having a plate shape. Alternatively, the forming mold may be a tool to be fitted to a container, for example, a forming mold having a mechanism via which the forming mold can cover the outer periphery of the upper surface of the container. The material of the main body of the mold is not limited to any particular one. It is preferable to employ, as the material of the main body, a material that would not change its property even when coming into contact with a liquid medium having not been solidified or gelated yet or with a solid medium obtained through solidification or gelation of the liquid medium. Since the main body is to be placed on the liquid medium, which has not been solidified or gelated yet and contains much moisture, it is preferable to use, as the material of the main body, a material that would not change its property even when water vapor adheres thereto and that can be retained on the plate in a stable manner. Examples of such a material encompass a stainless steel, plastic, and hard silicone. In order to discharge water vapor from the medium, the main body may be a board in which pores having a diameter of some millimeters are evenly formed.

In one aspect, the forming mold has a mechanism via which the protrusion is detachably attachable to a main body part. With this, it is possible to freely set the at least one protrusion at a desired position so as to form a fall-in cavity suitable for the purpose of use of the plate and the mode in which the plate is to be used. The attachment/detachment can be carried out by a method in which the protrusion is inserted or a method in which the protrusion is attached or detached with use of a magnet, for example. The method in which the protrusion is inserted can be carried out by the following manner. That is, holes are formed in the main body of the forming mold, and an insertion part such as a bar is provided to the at least one protrusion. Then, the insertion part is inserted into a desired one of the holes. Meanwhile, the method involving use of a magnet can be carried out by the following manner. That is, magnets may be provided to the main body of the forming mold and to an upper surface of the protrusion. Alternatively, a magnet may be provided to one of the main body of the forming mold and the upper surface of the protrusion, and a metal piece may be provided to the other. This makes it possible to position the protrusion at the desired position of the main body of the forming mold. In another aspect, the protrusion to be attached is not limited to a protrusion of a fixed shape and a fixed size. Alternatively, it is possible to employ protrusions that have different shapes and different sizes and that are designed to be freely set at desired positions. In addition, by adjusting the height(s) of the protrusion(s), it is possible to adjust the depth(s) of a fall-in cavity(ies) to be formed in the solid medium.

Furthermore, a clip-like fixing pin for pinching an upper part of a side wall of the container so as to fix the forming mold to the container may also be configured to be detachably attachable to the main body part. In addition, in order that the clip-like fixing pin can be fixed to the plate without damaging the plate, both distal ends of the fixing pin may respectively have nonslip caps attached thereto. The nonslip caps are each made of a material having a high coefficient of friction and elasticity, such as a silicone resin. The main body part is not limited to the plate-shaped member, provided that it can be fixed to the upper part of the container and it can accept the protrusion provided thereto. For example, the main body may be a member in another form, e.g., a lattice-shaped member or a bar-shaped member.

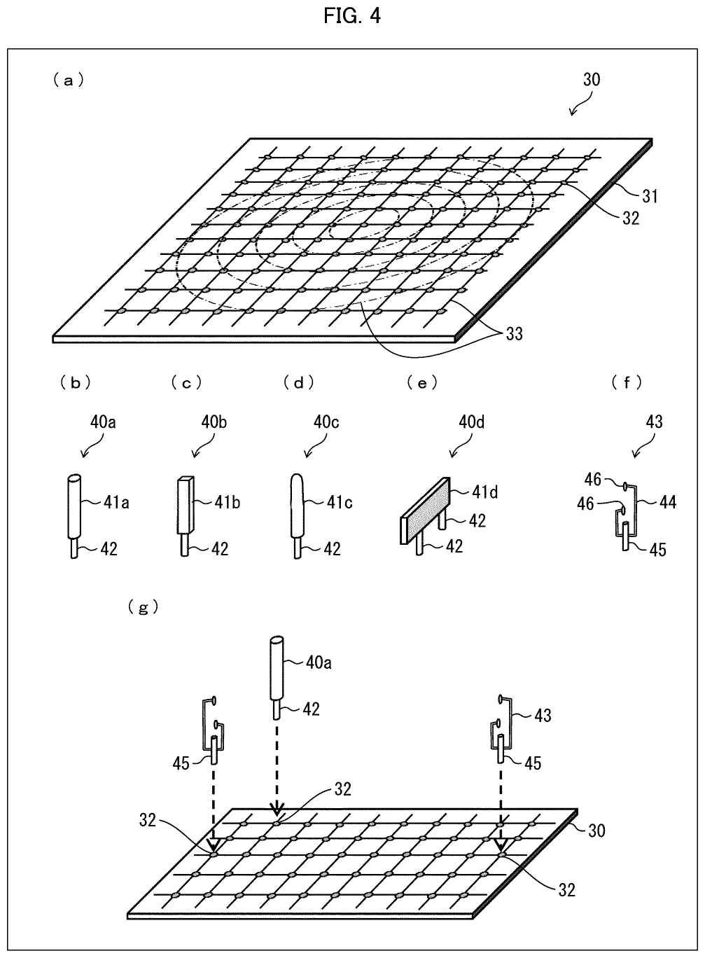

FIG. 4 illustrates examples of parts of a plate-shaped forming mold. (a) of FIG. 4 is a perspective view of an appearance of a plate-shaped main body part 30. (b) to (e) of FIG. 4 are perspective views of appearances of protrusions 40a to 40d (in cases where it is not necessary to distinguish the protrusions 40a to 40d from one another, the protrusions 40a to 40d will each be referred to as a “protrusion 40”). (f) of FIG. 4 is a perspective view of an appearance of a clip-like fixing pin 43.

As shown in (a) of FIG. 4, the main body part 30 includes a plate-shaped member 31 having a plurality of insertion holes 32 which are provided in a lattice pattern and with which a protrusion(s) 40 is detachably attachable to the main body part 30. In order that the protrusions 40 can be easily set on a straight line or concentrically, the plate-shaped member 31 may have a line 33 printed or formed thereon as a guide.

As shown in (b) to (e) of FIG. 4, each protrusion 40 includes (i) an insertion part 42 that is to be inserted into one of the insertion hole 32 so that the protrusion 40 is set to the main body part 30 and (ii) one of protrusion main bodies 41a to 41d (in cases where it is not necessary to distinguish the protrusion main bodies 41a to 41d from one another, the protrusion main bodies 41a to 41d will each be referred to as a “protrusion main body 41”) that is provided at a side of the insertion part 42 which side is opposite to a side being to be inserted into the one of the insertion holes 32, that is used to form a fall-in cavity, and that has a shape corresponding to the shape of the fall-in cavity. The protrusion 40a shown in (b) of FIG. 4 is a protrusion used to form a fall-in cavity having a circular shape with a flat bottom, and has the protrusion main body 41a having a cylindrical shape with a flat distal end. The protrusion 40b shown in (c) of FIG. 4 is a protrusion used to form a fall-in cavity having a rectangular shape with a flat bottom, and has the protrusion main body 41b having a rectangular column shape with a flat distal end. The protrusion 40c shown in (d) of FIG. 4 is a protrusion used to form a fall-in cavity having a circular shape with a round bottom, and has the protrusion main body 41c having a cylindrical shape with a round distal end. The protrusion 40d shown in (e) of FIG. 4 is a protrusion used to form a fall-in cavity having a rectangular shape with a flat bottom and having one long side like a groove, and has the protrusion main body 41d having a planer shape with a flat distal end. In order that the protrusion 40d can be attached to the main body part 30 in a stable manner, the protrusion 40d has a plurality of insertion parts 42.

As shown in (f) of FIG. 4, the fixing pin 43 includes an insertion part 45 that is to be inserted into one of the insertion holes 32 so that the fixing pin 43 is attached to the main body part 30 and a clip part 44 that is provided at a side of the insertion part 45 which side is opposite to a side being to be inserted into the one of the insertion holes 32 and that is used to pinch the side wall of the container. The clip part 44 has distal ends respectively provided with nonslip caps 46 for securely fixing the clip part 44 to the plate without damaging the plate.

(g) of FIG. 4 illustrates a state in which the protrusion 40a and the fixing pin 43 are to be attached to the main body part 30. As shown in (g) of FIG. 4, according to the size of the container, the number of fall-in cavities, the positions and shapes of the fall-in cavity(ies), and/or the like, a desired protrusion 40 is inserted into a desired one of the insertion holes 32 so as to be fixed thereto, and the fixing pins 43 are inserted to desired ones of the insertion holes 32 suitable for fixation to the container. Consequently, a desired forming mold can be obtained.

In the example shown in (g) of FIG. 4, the forming mold is fixed to the container by the method according to which the clip-like fixing pins are used to fix the plate-shaped main body part of the forming mold to the upper part of the container while causing the plate-shaped main body part of the forming mold to be supported by the upper part of the side wall of the container. However, this is not limitative.

Next, the following will explain the method for forming a fall-in cavity with use of the forming mold.

First, the forming mold is placed on the upper surface of the container. Subsequently, a liquid medium is poured into the container, and is then solidified (gelated). After solidification, the forming mold is gently removed. Consequently, a fall-in cavity is formed at a position where the protrusion of the forming mold resided. Alternatively, a liquid medium that is not solidified yet may be poured into the container, and then the forming mold may be placed on the upper surface of the container. Thereafter, the medium thus poured may be solidified.

FIG. 5 illustrates an example of a fit-in type forming mold. (a) of FIG. 5 is a top view of a fit-in type forming mold 50 used to produce a nematode trap plate in accordance with an embodiment, and (b) of FIG. 5 is a perspective view of a cross section of the forming mold 50. The example shown in FIG. 5 shows the forming mold designed to be used by being fitted into an upper surface of a commercially-available circular plastic dish. In the present embodiment, a side of the forming mold which side faces the container when the forming mold 50 is placed to cover the upper surface of the container is described as an upper surface side of the fit-in type forming mold.

As shown in (a) and (b) of FIG. 5, the forming mold 50 has an annular structure in which a main body outer frame (main body) 51, a fit-in part 52, and a protrusion 53, each of which has a shape corresponding to the shape of the outer periphery of the upper surface of the container, are provided in this order from the outer side. The main body outer frame 51 and the protrusion 53 are integrated with each other. The fit-in part 52 is a groove defined by the main body outer frame 51 and the protrusion 53 at a location between the main body outer frame 51 and the protrusion 53. The forming mold 50 is placed to cover the upper surface of the container, and the wall of the container is fitted into the fit-in part 52. Then, in a state where the container and the forming mold 50 are arranged in parallel with each other, a solid medium is formed. With this, at a position where the protrusion 53 resides, an annular, moat-like fall-in cavity is formed. Needless to say, the fall-in cavity is not limited to the one formed along the outer periphery of the container so as to have a moat-like form. Alternatively, the fall-in cavity may be circular, rectangular, or the like, as stated above. In this case, in accordance with the position of a fall-in cavity to be formed, a protrusion 53 having a desired shape and a desired size can be formed on an upper surface of a member horizontally extending from a fit-in part 52 toward the center of the annular shape. The present embodiment deals with the forming mold designed to be used by being fitted into the upper surface of the circular dish, and therefore the forming mold 50 has the annular structure. However, the outer profile of the forming mold 50 can vary depending on the shape of a container to be used. Since the forming mold comes into contact with the liquid medium that is not solidified or gelated yet and contains much moisture, the forming mold is preferably made of a material that would not change its property even when water vapor adheres thereto and that can be retained on the container in a stable manner. Examples of such a material encompass a stainless steel, plastic, and hard silicone. In order to discharge water vapor from the medium, the mold may the one in which pores having a diameter of some millimeters are evenly formed.

(Third Forming Method)

Next, with reference to FIG. 6, the following will describe the method for forming a fall-in cavity by forming a solid medium in a container in which a tubular container, which is to be turned into a fall-in cavity, is fixed to a hole formed in advance on the bottom surface of the container. First, the container to be used in this method will be explained. The description here explains the container in the form of a circular dish.

(a) of FIG. 6 is a top view of a container 10a to be used in this method, and (b) of FIG. 6 is a cross-sectional view taken along the broken line B-B in (a) of FIG. 6. As shown in (a) and (b) of FIG. 6, the container 10a has a bottom surface 5 having through-holes 4a and 4b at positions corresponding to the positions where fall-in cavities are to be formed. In FIG. 6, in order to form the plate 1 shown in FIG. 1, the through holes 4a and 4b are formed at positions where the through holes 4a and 4b respectively overlap the fall-in cavities 2a and 2b of the plate 1. However, the position(s) and the number of through holes may be appropriately set in accordance with the position(s) and the fall-in cavity(ies) to be formed. As will be described in detail later, the through holes 4a and 4b are parts into which tubular containers, which will be turned into fall-in cavities, are to be inserted. The container 10a is a raised-bottom container having a space below the bottom surface 5 in order to avoid a situation in which lower parts of the tubular containers having been inserted thereto come into contact with a table or the like on which the container 10a is placed.

The container 10a is identical to the container explained in the above-described section “(Nematode trap plate)” in terms of the material, the shape, and the like, except that the container 10a has a through hole on its bottom surface. The container 10a shown in FIG. 6 is a raised-bottom container. However, this is not limitative. Alternatively, the container 10a may be any container, provided that it has a structure in which a lower part of a tubular container having been inserted thereto does not come into contact with a table or the like on which the container is placed. For example, instead of the structure in which a space exists below the entire lower side of the bottom surface, the container may be structured to have a space only below a part of the lower side into which part a tubular container is to be inserted. Specifically, the container may be structured to have, on its bottom surface, one or more projections protruding upward and having through holes formed therein.

Next, the following will describe a method for forming a fall-in cavity in the plate with use of the container 10a.

First, tubes 6a and 6b (in cases where it is not necessary to distinguish the tubes 6a and 6b from each other, the tubes 6a and 6b will each be referred to as a “tube 6”), which are to be ultimately turned into fall-in cavities, are inserted into the through holes 4a and 4b formed on the bottom surface 5 of the container 10a (in cases where it is not necessary to distinguish the through holes 4a and 4b from each other, the through holes 4a and 4b will each be referred to as a “through hole 4”). Each tube 6 is a tubular container that has a bottom at its lower end and that is opened at its upper end. After the tubes 6 are inserted, a liquid medium that is not solidified or gelated yet is poured. Thus, in order to prevent leakage of the medium through a gap, each of the tubes 6 preferably has an outer contour coinciding with the shape of each through hole 4. Alternatively, parts functioning as stoppers, such as O-rings, may be attached between the through holes 4 and the respective tubes 6 so as to prevent falling-off of the tubes 6. Each of the tubes 6 may be a tube including, as a basic material, a material having a high coefficient of friction and elasticity, such as a silicone resin. Alternatively, each of the tubes 6 may be a tube coated with a silicone resin and/or the like.

Next, a liquid medium is poured into the container, and then is solidified or gelated to form a solid medium 11 ((d) of FIG. 6). In this process, the surface of the medium is set so as to be equal to or lower in height than the openings at the upper ends of the tubes 6. In order to prevent the medium from entering the tubes 6, the surface of the medium is preferably set so as to be lower in height than the openings at the upper ends of the tubes 6. Alternatively, in order to prevent the medium from entering the tubes 6, a detachable lid may be placed to cover the tubes 6.

After the solid medium 11 is formed, the tubes 6 are pushed down toward the bottom so that the upper ends of the tubes 6 are aligned in height with the surface of the solid medium 11. Consequently, recesses (fall-in cavities) are formed in the surface of the solid medium ((e) of FIG. 6). Thus, with this method, the tubes 6 embedded in the solid medium function as fall-in cavities.

In the nematode trap plate produced by this method, the tubes 6, which have been turned into the fall-in cavities, can be pushed up from the bottom surface 5 of the container 10a after the test, so that the tubes 6 are removed from the nematode trap plate. In this manner, it is possible to easily collect the nematodes caught in the tubes 6. Therefore, this method is suitably employed in a case where it is necessary to collect, after the test, the nematodes having been caught in the fall-in cavities.

The fall-in cavity having a moat-like structure can be formed by carrying out the following procedure, not by a certain method for forming a fall-in cavity. That is, a solid medium to be used as an island inside the moat is first formed by hollowing out a part of another solid medium, a fall-in cavity is then formed by any of the above-described methods, and thereafter the solid medium that is to be used as the island is positioned inside the fall-in cavity. Shortly, a solid medium to be used as an island inside the moat may be formed by hollowing out a part of another solid medium, and may be positioned inside a fall-in cavity formed by any of the above-described methods. Another solid medium from which the island-like solid medium is to be formed may be a solid medium equal or different in composition compared to the solid medium in the nematode trap plate on which the island-like solid medium is to be placed. In order to count the number of nematodes after the test, the island-like solid medium positioned inside the fall-in cavity may be removed.

[Method for Evaluating Response of Nematodes to Test Subject]

The following will describe a method in accordance with an embodiment for evaluating a response of nematodes with use of the above-described nematode trap plate. Described here is a method for supplying the test subject to the nematode trap plate and evaluating a response of the nematodes. Note that the method in accordance with the embodiment for evaluating a response of the nematodes with use of the nematode trap plate (PASS plate) can also be called “PASS method”.

(Preparation of Nematodes)

The nematodes to be used in the test are cultured in advance by a method suitable for the nematodes to be used, for example, a culturing method to be carried out on a certain solid medium or in a culture solution. The description here deals with a case where the nematodes to be used are C. elegans and are cultured in advance on an agar plate. However, this is not limitative. After the culturing, a buffer solution is poured onto the agar plate on which the nematodes have been cultured. In response to this, some nematodes start swimming in the solution. These nematodes are collected by, e.g., sucking with a pipette, and then are transferred to a tube for centrifugation. After leaving the nematodes in the tube for several minutes, an upper layer of the buffer solution which upper layer contains impurities such as Escherichia coli is removed. Then, a new buffer solution is added thereto to wash the nematodes, and an upper layer of the buffer solution is removed again. By repeating this process two or three times, a percentage of impurities that are not the nematodes is reduced. The kind, developmental stage, sex, and the like of the nematodes to be used in the test may be selected in accordance with the purpose of the test.

(Preparation of Test Plate)

1. Test Plate for Olfaction Test

The following will describe one example of a method for preparing a test plate used to carry out an olfaction test. In the example described below, a nematode trap plate for test has a fall-in cavity within which or around which a test subject is supplied and which is filled with a liquid. However, this is not limitative. That is, the present invention does not exclude use of nematode trap plates having other forms, e.g., a nematode trap plate having a fall-in cavity and a test subject supplying hole into which a test subject is to be supplied. Here, the test subject for the olfaction test can be, e.g., (a) a test subject itself that has become an odorant as a result of being diffused into a gas phase or (b) a substance that has become an odorant as a result of being released to a gas phase from a test subject in which the substance is originally contained.

The method for supplying the test subject may be a method of dripping the test subject into the fall-in cavity or onto an area around the fall-in cavity or a method of applying the test subject to the area around the fall-in cavity.

The fall-in cavity is filled with a liquid prior to the test. With the fall-in cavity filled with the liquid, it is possible to prevent the nematodes caught in the fall-in cavity from escaping from the fall-in cavity. In order that the nematodes having reached the fall-in cavity are caught, that is, trapped by the liquid in the fall-in cavity, it is preferable that the fall-in cavity be fully filled with the liquid, i.e., that the surface of the liquid reach the surface of the solid medium. In a case where relatively small nematodes are used, when the nematodes reach the fall-in cavity and come into contact with the surface of the liquid, the nematodes are drawn into the liquid by surface tension of the liquid. For example, in a case where C. elegans is used, when approximately one fifths of the body comes into contact with the surface of the liquid, C. elegans is drawn into the liquid.

Selectable as the liquid to be filled in the fall-in cavity is a liquid with which the test subject is suitably diluted and with which the test subject is suitably diffused into a gas phase. Examples of such a liquid encompass water (including ultrapure water), a physiological saline solution, a buffer solution, and ethanol. The present invention is not limited to the configuration in which the fall-in cavity is filled with the liquid in advance and the test subject is dripped thereto. Alternatively, an undiluted solution of the test subject or a diluted solution thereof may be poured into the fall-in cavity such that the fall-in cavity is filled with the liquid simultaneously with supplying of the test subject. With either of the above cases, the liquid to be filled in the fall-in cavity desirably has a temperature identical to that of a part of the solid medium which part is around the fall-in cavity.

In the test involving use of the control fall-in cavity, the control fall-in cavity is identical to the fall-in cavity into which the test subject is supplied in terms of all kinds of liquids, the capacity (volume), the temperature, and the pouring timing, except that the control fall-in cavity does not contain the test subject.

Note that, even in a case where the number of fall-in cavities is two or more, the control fall-in cavity may not necessarily be provided. For example, in (a) a comparative test carried out to rank attraction responses to two or more different concentrations of a single chemical substance in order, (b) a comparative test carried out to make a comparison between responses to two or more different kinds of chemical substances, and (c) a comparative test carried out to make a comparison between responses to two or more kinds of chemical substances having different characteristics (e.g., an odorant and a taste substance), a fall-in cavity filled with a standard solution serving as a control may not be provided and all fall-in cavities may be used for the test.

The test plate used in the olfaction test may be subjected not only to the above-described processing but also to processing for regulating the releasing or diffusion rate of an odorant from a liquid that is to be tested (hereinafter, such a liquid may sometimes be referred to as a “test solution”) which liquid is filled in the fall-in cavity and contains the test subject. By regulating the releasing or diffusion rate of the odorant, it is possible to optimize the odor sensitivity of the nematodes. If the odorant released from the test solution filled in the fall-in cavity is saturated in a space inside the nematode trap plate, the nematodes cannot exhibit taxis, i.e., selective migration toward the odor source or selective migration away from the odor source. Similarly to the space, the state of the odorant adhered to the surface of the solid medium may also involves difficulty in carrying out the test. That is, it becomes difficult to carry out the test, if the odorant is adhered to the surface of the solid medium at a density higher than the density at which a density gradient can be maintained. Thus, in a case where the odorant to be tested is a substance easily diffused, a chemical agent for reducing the rate of releasing of molecules from the surface of the test solution filled in the fall-in cavity is added to the test solution in the fall-in cavity. Meanwhile, in a case where the odorant is a substance that is hardly evaporated, a chemical agent that can serve as a sensitizer is added to the test solution filled in the fall-in cavity. In the test involving use of a control fall-in cavity, the chemical agent added to the test solution is also added to the standard solution filled in the control fall-in cavity in the same amount as that added to the test solution. Usable as the chemical agent for reducing the rate of releasing of molecules is a conventionally-known substance having an evaporation inhibiting effect. Usable as the sensitizer is a conventionally-known substance having a gas diffusion effect. With this, it is possible to regulate the amount, rate, and range of diffusion of the odorant to be tested, thereby making it possible to regulate the sensitivity of odor and/or the like in the nematodes. Thanks to the configuration that can regulate the releasing or diffusion rate of the odorant, it is possible to optimize (particularly, minimize) the size of the nematode trap plate.

2. Test Plate for Gustation Test

The following will describe one example of a method for preparing a test plate used to carry out a gustation test. In a nematode trap plate for test in the example described below, a test subject is supplied to an area around a fall-in cavity so as to form a concentration gradient therein, and the fall-in cavity is filled with a liquid. However, this is not limitative. Here, the test subject for the gustation test can be, e.g., (a) a test subject itself that has become a taste substance or (b) a substance that has become a taste substance as a result of being released to a solid medium from a test subject in which the substance is originally contained.

First, a plug containing the test subject at a high concentration is prepared. Then, the plug thus prepared is put into the fall-in cavity in the nematode trap plate or is placed on the solid medium so as to cover the fall-in cavity. Consequently, the test subject is diffused from the plug into the solid medium, so that a concentration gradient of the test subject is formed on the solid medium concentrically about the fall-in cavity. Here, the plug means a small piece prepared by hollowing out a part of a plate-shaped solid medium containing the test subject and having a desired thickness.

In a case where the fall-in cavity is to be formed by carrying out hollowing-out at a timing immediately before the test, the hollowing-out may be carried out to form the fall-in cavity after the concentration gradient of the test subject is formed in the solid medium. Specifically, the plug containing the test subject is allowed to stand at a position on the solid medium at which position the fall-in cavity is to be formed. Consequently, the concentration gradient of the test subject is formed concentrically about the position where the fall-in cavity is to be formed. After the plug is removed, the fall-in cavity may be formed by hollowing out a part of the solid medium at that position. Consequently, the concentration gradient of the test subject is formed concentrically about the fall-in cavity.

A certain duration for allowing the plug to stand can be set as appropriate in accordance with the size and shape of the plate, the kind of the solid medium, the size and shape of the fall-in cavity, the kind and concentration of the test subject, and the like.

The size and shape of the plug can be set as appropriate in accordance with the size and shape of the fall-in cavity, the kind of the test subject, and the specific method for testing. In one example, in a case where a circular dish having an inner diameter of 5.0 cm to 10.0 cm is used as the container of the plate and the thickness of the solid medium is set so as to fall within a range from approximately 5 mm to approximately 2 cm, it is possible to employ a plug having a thickness of 3 mm to 1 cm, a size of some mm to approximately 1 cm square or a diameter of some mm to approximately 1 cm.

Even in a case where the test subject is supplied via the plug, the fall-in cavity is filled with a liquid at the time of starting the test. For example, after the plug is removed from the solid medium, the liquid may be poured into the fall-in cavity before the test is started. The liquid can be the same liquid as that used in the above-described preparation of the test plate for the olfaction test.

In the test involving use of the control fall-in cavity, the control fall-in cavity is identical to the fall-in cavity into which the test subject is to be supplied in terms of all kinds of liquids, the capacity (volume), the temperature, and the pouring timing, except that the control fall-in cavity does not contain the test subject. In addition, the control fall-in cavity is preferably subjected to the same processing with use of a plug identical to the plug used to form the concentration gradient of the test subject in terms of the composition, size, and shape, except that the plug for the control fall-in cavity does not contain the test subject.

Also in the gustation test, in a case where the number of fall-in cavities is two or more, the control fall-in cavity may not be necessarily provided.

The description above has dealt with the case where the test subject is supplied via the plug. Alternatively, the test subject may be caused to penetrate into the solid medium by a method of dripping a solution containing the test subject onto an area of the plate which area is around a part that is to be turned into a fall-in cavity and drying the solution thus dripped. Further alternatively, a method of filling the fall-in cavity with a solution containing a taste substance may be employed. Still further alternatively, the test subject may be supplied into the fall-in cavity by a method of putting, into the fall-in cavity, a plug containing the test subject and filling the fall-in cavity with a liquid.

(Supplying of Nematodes)

After the test plate to which the test subject has been supplied is prepared, the nematodes are supplied to the plate, and the test is started.

Typically, the supplying of the nematodes can be carried out by dripping a liquid containing the nematodes having been washed onto a certain position of the plate to which the test subject has been supplied and by wiping away the liquid to remove the liquid.

The number of nematodes to be supplied may be set as appropriate in accordance with the number of fall-in cavities, the size(s) of the fall-in cavity(ies), the number counting method, the purpose of the test, and/or the like. For example, in a case of using C. elegans as the nematodes and a plate having two fall-in cavities each having a diameter of 5 mm and a depth of 4 mm, the number of nematodes is approximately 100 to 1000. In a case of employing the later-described method of counting the number of individual nematodes by individually recognizing the nematodes, the number of nematodes to be supplied is preferably set at a number with which the nematodes in a certain single fall-in cavity can be visually observed even if all of the nematodes having been supplied are caught in the certain single fall-in cavity. That is, in such a case, the number of nematodes to be supplied is preferably approximately 100. Meanwhile, in a case of employing the later-described method of measuring another factor that correlates with the number of nematodes and calculating the number of nematodes, the number of nematodes to be supplied can be set to approximately 1000.

The position to which the nematodes are to be supplied may be selected as appropriate in accordance with the purpose of the test. Preferably, the position to which the nematodes are to be supplied is a position separated away from the fall-in cavity. In a case where the test is conducted with use of a plurality of fall-in cavities, the position to which the nematodes are to be supplied is typically a position equally separated away from all the fall-in cavities. Thus, in one aspect, a plate can be configured to have (a) two or more fall-in cavities formed in or in the vicinity of the outer periphery of the solid medium and (b) a center part in which no fall-in cavity is formed and to which the nematodes can be supplied.

The present invention is not limited to the configuration in which the nematodes are supplied to one position. Alternatively, for example, in a case of employing a rectangular plate having a plurality of fall-in cavities provided along the long sides of the plate, the nematodes may be supplied onto a straight line that passes through the center of the plate and that is in parallel with the long sides.

When the nematodes are supplied in the manner described above and then the liquid supplied together with the nematodes is removed, the nematodes start moving freely over the solid medium of the plate. Thus, immediately after the liquid is removed, the cover is placed to cover the plate.

(Test Conditions)

The test is conducted by leaving the plate until a certain duration elapses after the cover is placed to cover the plate. There is no particular limitation on the environment in which the plate is left. However, in order to prevent a situation in which the result of the test is affected by the environmental conditions, the conditions such as the temperature of the place where the plate is left and the presence/absence of light shielding may be determined as appropriate in accordance with the purpose of the test, the kinds of the test subject and the nematodes, and/or the like. A simple way to leave the plate with light shielding at a given temperature is to leave the plate in (a) a cool incubator having a door without a light-transmitting part such as a window (b) a cool incubator having a door with a light-transmitting part having been subjected to a light-shielding treatment with an aluminum foil and/or the like. Alternatively, a vat made of a light impermeable material, such as a stainless steel, aluminum, or opaque plastic, may be placed to cover the plate.

(Obtaining Number of Nematode Individuals)

When the certain duration has elapsed, the test is ended. Then, the number of nematodes caught in the fall-in cavity is obtained.

The number of nematodes can be obtained by a method selected from the two methods, i.e., (i) the method of counting the number of nematodes in the fall-in cavity by individually recognizing the nematodes and (ii) the method of measuring another factor that correlates with the number of nematodes in the fall-in cavity and calculating the number of nematodes. These methods (i) and (ii) may be carried out through image analysis on a captured image of a target area, and may be carried out after the nematodes having been caught are collected. If visual observation is sufficiently possible, the number of nematodes in each fall-in cavity may be counted through microscopic observation. In a case where image capturing is carried out, the number counting by visual observation and the later-described counting through image analysis may be employed in combination. In a case where a light-transmitting cover is employed, an image of the nematodes in the fall-in cavity may be captured under a microscope with the plate being covered with the cover. Examples of another factor that correlates with the number of nematodes encompass a fluorescence intensity obtained in fluorescence observation, shading in a captured image, a light absorbance of the liquid that has been collected together with the nematodes and that contains the nematodes, an indicator that is a nucleic acid and a protein themselves extracted from the collected nematodes, and an indicator obtained by subjecting the nucleic acid and the protein to a treatment (e.g., an amplification treatment, an enzymatic reaction treatment). In some cases, the nematodes and liquid in the fall-in cavity may be transferred to another container shortly after the test. In this case, an image of the container to which the nematodes and liquid have been transferred may be captured, and image analysis may be carried out on the image. The following will describe some specific examples of the method of counting the number of individual nematodes. However, the present invention is not limited to them. Each of the methods (i) and (ii) may be used alone. Alternatively, two or more selected from the methods classified into (i) and (ii) described later may be used in combination.

[A. Method of counting individual nematodes by individually recognizing nematodes through image analysis]

In a case of employing this method, an image to be observed may be a bright field image or a fluorescence image.

(A-1) Image capturing: An image of the fall-in cavity having been used in the test under a microscope is captured with use of a recording device such as a digital camera, a video camera, or the like. In a case where the nematodes and liquid in the fall-in cavity have been transferred to another container after the test, an image of the liquid in that another container, in place of the fall-in cavity, is captured with use of the recording device. For example, an image of the inside of that another container into which the nematodes and liquid in the fall-in cavity have been collected is captured through the bottom surface with use of the recording device.

(A-2) Pre-processing: The still image or moving image captured in (A-1) above is subjected to filter processing (low-pass filter, smoothing filter, and/or the like) and then to binarization (conversion into monochrome photographic data), a contrast enhancement treatment, and/or the like. In a case where the image is to be subjected to binarization, the image is basically processed so that the nematodes, which are to be measured, are represented in black.

(A-3) Extraction of feature quantity: On the basis of the area of the nematodes that are to be recognized, an extraction threshold (e.g., a threshold in terms of the number of pixels) is set, and shading (noises) that does not correspond to the nematodes are removed. Then, the contours, the center lines, the histograms of oriented gradients (HOG) feature quantities, the areas, and/or the like of the nematodes are calculated. With this process, it is possible to remove, as noises, everything other than the inside of the fall-in cavity. In addition, it is also possible to limit, at an early stage, an image analysis target to the fall-in cavity by a method of setting the size of an analysis target area (i.e., setting a uniform magnification of image capturing) and/or the like in advance. In addition, deep leaning using artificial neural networks may be employed to discriminate the nematodes and the noises from each other without setting the threshold and/or the like.

(A-4) Individual recognition and individual separation: The nematodes are recognized by using an algorithm of machine learning, such as artificial neural networks and/or support vector machine, or pattern matching. In a two-dimensional image representing the inside of the fall-in cavity, which is a three-dimensional space, two or more individuals are superimposed to each other in many cases. In such a case, particularly, pattern matching and/or the like is applied to separate the two or more individuals from each other to recognize them as different individuals.

(A-5) Counting: The individuals extracted, in (A-4) above, as the nematode individuals from the image are numbered, and the total number of nematodes in the fall-in cavity is obtained.

The processes in (A-1) to (A-5) above may be carried out manually or fully automated, and can be realized by generally-known algorithms.

[B. Method of measuring another factor that correlates with the number of nematodes and calculating the number of nematode individuals through image analysis]

Examples of another factor that correlates with the number of nematodes in image analysis encompass (B1) a method involving use of shading of a group of nematodes and (B2) a method involving use of the quantity of light emitted from the nematodes. The following will describe each of the methods. However, the present invention is not limited to them. Also in a case of employing this method, an image to be observed may be a bright field image or a fluorescence image.

(B1. Method involving use of shading of group of nematodes)

(B1-1) Pre-processing on image: The processes (A-1) to (A-3) described above are carried out in advance.

(B1-2) Calibration: Correspondence between the number of nematodes and the area of the image having been subjected to the image pre-processing (B 1-1) is obtained. For example, a nematode(s) of the same kind as the nematodes used for the test is/are introduced into the fall-in cavity while the number of nematodes to be introduced is increased in several steps in this order: one, five, ten, 20, 30, 40, and 50. Then, an image of the fall-in cavity is captured at each step. For the respective numbers of individuals, the areas of the images corresponding to the nematode individual(s) are calculated. Then, a standard curve is drawn. On the basis of the standard curve, correspondence data is created that is used to estimate the number of nematodes caught in the target range (fall-in cavity) from the area of a part of the image which part corresponds to the nematode individuals.

(B1-3) Counting: For the target image having been subjected to the pre-processing (B1-1), the number of nematodes within the analysis target range is calculated from the area (the number of pixels) of the target image on the basis of the data (B1-2).

(B2. Method involving use of quantity of light)

There is no limitation on the kind and intensity of the light used in this method. Thus, the light is not limited to fluorescence emitted from a recombinant organism into which a fluorescent probe or the like is incorporated, and may be autofluorescence. Alternatively, the light may be light derived from coloration or light emission caused by using a protein, a nucleic acid, or a certain kind of endogenous enzyme activity. A transgenic nematode which has a fluorescent probe (e.g., a green fluorescent protein) incorporated therein and the whole of which can be constantly under fluorescence observation may be, for example, the HBR4 strain of the nematode C. elegans. The HBR4 strain has a fluorescent probe incorporated in its body-wall muscle cells, which are used to swim in a liquid. With the HBR4 strain, a concentration of calcium ions discharged upon contraction of the body-wall muscle cells can be indirectly measured as a fluorescence intensity. Preferably, the transgenic nematode is the one whose response to the test subject is identical to that of a wild-type nematode. The following description will deal with, as an example, a case of employing a transgenic nematode having a fluorescent probe incorporated therein.

(B2-1) Image capturing: Under a fluorescence microscope, an image of an analysis target area, that is, the inside of a fall-in cavity or the inside of a container into which the content of the fall-in cavity has been transferred is captured with a recording device such as a digital camera or a video camera. The conditions of the fluorescence observation and image capturing, such as a wavelength of excitation light and a filter, may be selected in accordance with, e.g., specifications of a transgenic nematode and a microscope to be used, for the purpose of enhancing the fluorescence intensity in a stable manner. Since the image-capturing area is limited to the inside of the fall-in cavity or the inside of the container into which the content of the fall-in cavity has been transferred, a high-resolution camera is not necessarily needed. A commercially-available digital camera is suitably applicable.

(B2-2) Calibration: Correspondence between the number of nematodes and the fluorescence intensity in the still image or moving image captured in (B2-1) is obtained. For example, a nematode(s) of the same kind as the nematodes used for the test is/are introduced into the fall-in cavity while the number of nematodes to be introduced is increased in several steps in this order: one, five, ten, 20, 30, 40, and 50. Then, an image of the fall-in cavity is captured with a fluorescence microscope at each step. From the captured images, the total of the fluorescence intensities in the fall-in cavity is calculated. Then, a standard curve is drawn. This processing can be carried out with use of a self-made image analyzing program, an image analyzing program supplied by a third party with or without charge, or commercially-available image analyzing software (hereinafter, these may sometimes be collectively referred to as “image analyzing software, etc.”). On the basis of the result of the analysis, correspondence data is created that is used to estimate, from a fluorescence intensity, the number of nematodes caught in the target range (fall-in cavity). Carrying out this calibration once prior to the test make it possible to automatically derive, in a later test(s) in which nematodes of the same kind are used, the number of individuals from the fluorescence intensity of the image, without manually counting the number of nematodes each time of the test is conducted.

(B2-3) Counting: For the target image captured in (B2-1) above, the number of nematodes within the analysis target range is calculated from its fluorescence intensity on the basis of the data created in (B2-2).

[C. Method of collecting nematodes, measuring another factor that correlates with the number of nematodes, and calculating the number of individuals]

Examples of the method of collecting nematodes and then measuring another factor that correlates with the number of nematodes encompass the method of carrying out measurement based on a light absorbance of the liquid that has been collected together with the nematodes and that contains the nematodes, the method of carrying out measurement on the basis of an indicator that is a nucleic acid and a protein themselves extracted from the collected nematodes or an indicator obtained by subjecting the nucleic acid and the protein to a treatment, and the method of carrying out measurement on the basis of a fluorescence intensity obtained through fluorescence observation on nematodes having been collected, as described above. As one example thereof, a method involving use of DNA of nematodes is described. For example, pine wilt disease is detected (diagnosed) by quick diagnosis (required duration: approximately 60 minutes) employing the principle according to which DNA of pine wood nematodes intruded into a wood piece is amplified by Long-mediated isothermal amplification (LAMP) method. According to this detection method, if the test solution obtained after the DNA extraction and amplification treatment exhibits a green fluorescent color, this means positive and the existence of pine wood nematodes is confirmed. By making use of this method, it is possible to estimate (identify) the number of individuals from the amount of DNA of the nematodes caught in the fall-in cavity. The following will describe typical procedures therefor.

First, the nematodes caught in the fall-in cavity are collected into a container, for example, a tube, together the liquid filled in the fall-in cavity. In a case of employing a plate into which a detachable tube is embedded in advance so as to form a fall-in cavity, the tube may be detached from the plate after the nematodes are caught, so that the nematodes are collected. The sample containing the collected nematodes is subjected to DNA extraction and an amplification treatment. Relation between the DNA amplification time period and the number of individuals required until the test solution emits certain green fluorescence is studied in advance, and the total number of nematodes caught in the fall-in cavity is determined based on the relation thus studied. Alternatively, while a DNA amplification time period is fixed, relation between the intensity or the like of green fluorescence observed after the time period has elapsed and the number of individuals may be studied in advance, and the total number of nematodes caught in the fall-in cavity may be determined based on the relation thus studied.

[D. Implementation of image analyzing software, etc.]

The image analyzing software, etc. used in the image analysis after image capturing and the number counting after the image analysis among the processes included in the above-described [A. Method of counting individual nematodes by individually recognizing nematodes through image analysis], [B. Method of measuring another factor that correlates with the number of nematodes and calculating the number of nematode individuals through image analysis], and

[C. Method of collecting nematodes, measuring another factor that correlates with the number of nematodes, and calculating the number of individuals] are typically installed in a general-purpose personal computer or a work station (hereinafter, the general-purpose personal computer and the work station may sometimes be referred to simply as a “computer” together) and used. Alternatively, the image analyzing software, etc. may be installed in a mobile terminal (e.g., a smartphone) including an operating system for a mobile device and used.

The computer or mobile terminal (hereinafter, the computer and the mobile terminal may sometimes be referred to simply as a “medium” together) in which the image analyzing software, etc. are to be installed is desirably the one having a radio communication function. In a case where the medium is equipped with a camera, the image capturing for automatic counting may be carried out with the camera of the medium, in place of the recording device such as the digital camera or the video camera. With the still image or moving image thus captured or the still image or moving image taken into the medium after being captured by the recording device such as the digital camera or the video camera, the image analysis and the number counting of the nematodes are carried out via the medium. The result of the number counting is indicated on the medium via which the number counting is carried out, or is transmitted to another medium so as to be indicated thereon. Even in a case where the number of nematodes in the fall-in cavity is counted through visual observation of the captured image, the whole of or a part of this process and the result of analysis can be indicated with use of the medium.

Note that, even in a case where automatic processing involving use of the image analyzing software, etc. is basically carried out, the number counting may be carried out through visual observation. That is, it is possible to carry out both the automatic processing and the visual observation as necessary. For example, there may be cases where the still image or moving image is difficult to be processed with the image analyzing software, etc., such as a case where the number of nematode individuals in the fall-in cavity is quite large or a case where the image is unclear. For such an image, the number counting through visual observation may also be carried out. That is, the processing with the image analyzing software, etc. and the counting through visual observation may be carried out in combination. In such a case, it is possible to employ work management software configured to automatically check the quality of an image and make a selection of a number counting method, specifically, to automatically extract only an image that is difficult to be analyzed with the image analyzing software, etc. and request to carry out number counting through visual observation, for example.

(Method for Evaluating Test Result) On the basis of the number of nematodes caught in the fall-in cavity which number has been obtained in the above-described manner, a response of the nematodes is evaluated. In the olfaction test or gustation test for evaluating a response of the nematodes to a chemical substance or the like, it is possible to evaluate the response on the basis of, e.g., a chemotaxis index (it may sometimes be abbreviated as “C.I.”). The chemotaxis index, which is used to evaluate the taxis of the test subject to the chemical substance or the like, is defined by in the following manner. That is, the number of nematodes caught in a control fall-in cavity is subtracted from the number of nematodes caught in a fall-in cavity for the test subject so that a difference is yielded, and the difference is divided by the total number of nematodes caught in all the fall-in cavities. In a case where there exist two or more fall-in cavities for the test subject, the total number of nematodes caught in these fall-in cavities is considered as “the number of nematodes caught in the fall-in cavity for the test subject”. Similarly, in a case where there exist two or more control fall-in cavities, the total number of nematodes caught in these control fall-in cavities is considered as “the number of nematodes caught in the control fall-in cavity”. Specifically, the chemotaxis index (C.I.) can be obtained by the following formula, where Ni is the total number of nematodes caught in the fall-in cavity for the test subject and N₂ is the total number of nematodes caught in the control fall-in cavity.

Chemotaxis index (C.I.)=(N₁−N₂)/(N₁+N₂).

In principle, a positive value means that, in a case of the olfaction test, the nematodes have been attracted to the odor of the test subject and that, in a case of the gustation test, the nematodes have been attracted to the taste of the test subject. Meanwhile, a negative value means that the nematodes have avoided the odor or taste of the test subject. Note that, if C.I. exhibits a negative value in a test involving use of a control fall-in cavity filled with an odored liquid, this may mean that the nematodes have been attracted to the odor of the liquid filled in the control fall-in cavity. Note also that, if C.I. exhibits a positive value in such a test, this may mean that the nematodes have avoided the odor of the liquid filled in the control fall-in cavity. Thus, the liquid to be used for dilution and the liquid to be filled in the control fall-in cavity should be selected in consideration of the above matter. Also in the warm-sensing test or the like, it is possible to evaluate an attraction or avoidance response to the test subject and the target to be tested, such as a temperature, on the basis of an index that is in accordance with C.I. used in the olfaction test and gustation test. In the warm-sensing test, a generally-known thermotaxis index, so-called TTX Index, can be used.

[Method for Evaluating Response of Nematodes to Coexisting Organism]

The following will describe a method in accordance with another embodiment for evaluating a response of nematodes with use of the above-described nematode trap plate. The present embodiment describes an aspect of an olfaction test in which an organism is used as a test subject that emits an odorant.

For people of agriculture and forestry, it is important to appropriately and efficiently exterminate agricultural pests. Plant-parasitic nematodes are parasitic on the roots, leaves, and the like of plants, and accordingly cause great damage such as withering of the roots and leaves. The following will describe one example of a test for electing an antagonistic or repellent organism against the plant-parasitic nematodes which test is designed to effectively exterminate the plant-parasitic nematodes with use of a predator or an antagonistic plant against the plant-parasitic nematodes. Specifically, a predator (e.g., tardigrade) or an antagonistic plant (e.g., Tagetes patula) against the plant-parasitic nematodes is used to examine whether or not the plant-parasitic nematodes can scent out the odor and/or the like emitted by the predator or the antagonistic plant and avoid the predator or the antagonistic plant. A most suitable coexisting organism of an agricultural plant is a nematode predator or an antagonistic plant that emits an order and/or the like that the plant-parasitic nematodes cannot sense out and easily get close thereto.

The following will describe an antagonistic organism election test in which a nematode predator is assumed. In this test, a nematode trap plate is prepared so as to have (a) a fall-in cavity for a test subject and a control fall-in cavity formed therein and (b) a recess for storing nematode predators (hereinafter, such a recess may sometimes be referred to as an “organism stand-by cavity”) formed in the vicinity of the fall-in cavity for the test subject. Into the organism stand-by cavity, tardigrades, which are predators of the plant-parasitic nematodes and which can be a candidate of an antagonistic organism, are introduced in advance. In a case of using the nematode predators (e.g., tardigrades) that cannot escape from the organism stand-by cavity, the organism stand-by cavity is not filled with a liquid. Meanwhile, the fall-in cavity for the test subject and the control fall-in cavity are each filled with a liquid. It is assumed that, if the plant-parasitic nematodes sense, as a repellent, the odor emitted by the nematode predators in the organism stand-by cavity, the plant-parasitic nematodes would be caught more in the control fall-in cavity than in the fall-in cavity, which is close to the organism stand-by cavity. In the test, first, the tardigrades are introduced into the organism stand-by cavity, and the fall-in cavities are filled with a liquid. Next, the plant-parasitic nematodes having been washed are supplied to the plate. Immediately after that, the plate is covered with a cover. Assuming that this point of time is a test start time, the plate is left under predetermined environmental conditions (e.g., a temperature) until a predetermined duration elapses. The number of plant-parasitic nematodes to be supplied varies depending on the number of fall-in cavities, the sizes of the fall-in cavities, the method of counting the number of the plant-parasitic nematodes, and the purpose of the test. Typically, the number of plant-parasitic nematodes to be supplied is approximately 100 to approximately 1000. For example, in a case where the number of nematodes is obtained by the above-described method of counting the number of individual nematodes by individually recognizing the nematodes, the number of plant-parasitic nematodes to be supplied is preferably set at a number with which the nematodes in a certain single fall-in cavity can be visually observed even if all of the nematodes having been supplied are caught in the certain single fall-in cavity. That is, in such a case, the number of nematodes to be supplied is preferably approximately 100. Meanwhile, in a case of employing the above-described method of measuring another factor that correlates with the number of nematodes and calculating the number of nematodes, the number of nematodes to be supplied can be set to approximately 1000.

The method of counting the number of plant-parasitic nematode individuals and the method for evaluating the result of the test that are to be carried out after the test is ended upon lapse of a predetermined duration may be carried out in the same manner as described above in the method for evaluating a response of the nematodes to the test subject.

[Method for Evaluating Response of Nematodes to Temperature]

Next, the following will describe a method in accordance with another embodiment for evaluating a response of nematodes with use of the above-described nematode trap plate. The description here deals with a warm-sensing test of evaluating a response of nematodes to a temperature with use of a nematode trap plate on which temperature adjustment has been carried out.

In the warm-sensing test, at the stage of preparation of the test plate, the temperature of the inside of a fall-in cavity or an area around the fall-in cavity is adjusted at a desired value, instead of supplying a test subject into the fall-in cavity or the area around the fall-in cavity. The temperature of the inside of the fall-in cavity or the area around the fall-in cavity is adjusted by the following method. That is, the nematode trap plate is placed on a temperature control device including a plate having a desired width and a temperature controller(s) that is/are disposed at a part of the plate, preferably at both ends of the plate, more preferably in the entire plate including its center and that can control the temperature into a desired value. Consequently, a temperature gradient is formed in the solid medium. In this process, the nematode trap plate may be placed on the temperature control device such that the fall-in cavity is located in a temperature range to be tested. As the temperature control device, a so-called thermoplate is suitably usable. However, the temperature control device is not limited to this aspect, provided that it is a device that can form a temperature gradient on a plane. In a case where a temperature lower than room temperature is to be considered, the plate is heated in advance to a temperature equal to or higher than the upper limit (e.g., 25° C.) of a temperature range to be tested, and is turned over before a liquid is filled in the fall-in cavity. Then, for example, a cylindrical bin containing glacial acetic acid is placed on the center of the fall-in cavity, so that a temperature gradient is formed concentrically. The container into which glacial acetic acid is put is not limited to the one having a circular bottom surface. The shape of the bottom surface of the container may be changed according to the purpose of the test. Use of glacial acetic acid having a melting point of 17° C. makes it possible to form a temperature gradient in which a temperature at a position close to the center of the fall-in cavity is 17° C. and a temperature at a position separated farthest from the center is 25° C. at maximum. In the present embodiment, it is desirable that the temperature of the liquid to be filled in each fall-in cavity be identical to the temperature of a part of the solid medium which part is around the fall-in cavity.

As described above, the nematodes are supplied in a state where the temperature adjustment has been carried out, and then the test is started. In the warm-sensing test, it is preferable that the adjusted temperature be kept during the testing duration.

The method for preparation of the nematodes, the method for counting the number of nematode individuals, and the method for evaluating the result of the test may be carried out in the same manner as described above in the method for evaluating a response of the nematodes to the test subject.

[Method for Evaluating Behaviors of Nematodes]

The above-described nematode trap plate can be used not only to evaluate a response of the nematodes to the test subject or the temperature, but also to evaluate the behaviors of the nematodes in order to evaluate the state and nature of the nematodes. For example, as described below, the nematode trap plate can also be used in an evaluation test in which a subject to be tested, such a certain test subject or a temperature, is not assumed, e.g., in an evaluation test for evaluating the stress degree of the nematodes and/or the motility of the nematodes. The following will describe a method in accordance with an embodiment for evaluating the behaviors of nematodes with use of the above-described nematode trap plate.

[Method for Evaluating Stress Degree of Nematodes]

The following will describe a method in accordance with an embodiment for evaluating the stress degree of nematodes with use of the above-described nematode trap plate. The present embodiment is to evaluate the stress degree of the nematodes while focusing on an aspect of a stress response of the nematodes, that is, a response of escaping toward an environment without food. The description below deals with a case of employing C. elegans as the nematodes. However, this is not limitative.

The nematodes are likely to remain in an environment in which sufficient food exists. However, when the nematodes are under a stress condition or the nematodes are subjected to external or internal stimulation(s) of a certain or more degree, the nematodes sometimes move away from the environment with food to seek or get close to an environment without food. That is, when Escherichia coli, which is food, is cultured in the center of a plastic dish filled with a solid medium and the nematodes are released thereon, the nematodes normally remain within the range in which E. coli is cultured. Under the stress condition, however, the nematodes may sometimes move away from the food range to get close to the edge of the plastic dish, particularly, to the inner wall of the plastic dish. One example of a stress degree evaluation test for evaluating the stress degree of the nematodes by making use of this nature will be described below.

The following will describe the stress degree evaluation test of the nematodes. This test uses the nematode trap plate, examples of which are shown in (e) and (f) of FIG. 2, having the fall-in cavity(ies) formed in the outer periphery of the solid medium. E. coli, which is food, is cultured in the center of the solid medium. Approximately 100 nematodes are released onto E. coli. Then, after a certain duration has elapsed, the number of nematodes caught in the fall-in cavity is counted. In the present embodiment, the liquid to be filled in the fall-in cavity may be any liquid, provided that it is odorless and does not have a difference in temperature from the solid medium. Examples of such a liquid encompass ultrapure water and a physiological saline solution. The number of nematodes caught in the fall-in cavity may be counted by the method of capturing an image of the inside of the fall-in cavity and counting the number of nematodes based on the image thus captured. If a problem of a large image-capturing range arises, the nematodes caught in the fall-in cavity may be collected into a tube or the like together with the liquid filled in the fall-in cavity and an image of the nematodes in the tube may be captured. If it is difficult to collect the nematodes because of, e.g., a small volume of the fall-in cavity, the nematodes that are not caught in the fall-in cavity and left on the solid medium may be collected with use of a platinum wire and/or the like, a liquid of the same kind as that filled in the fall-in cavity may be poured into the nematode trap plate, and then the nematodes floated up onto the nematode trap plate may be collected together with the liquid. The conditions, methods, and/or the like described for the above-described method for evaluating a response of the nematodes to the test subject may be applied to various other conditions such as the temperature condition and the light condition in the present embodiment.

The stress degree of the nematodes can be evaluated, for example, based on a value obtained by dividing the number of nematodes caught in the fall-in cavity by the total number of nematodes initially released.

[Method for Evaluating Motility of Nematodes]

The following will describe a method in accordance with an embodiment for evaluating the motility of nematodes with use of the above-described nematode trap plate. The present embodiment evaluates the motility of the nematodes based on the migration length of the nematodes. The description below deals with a case of employing C. elegans as the nematodes. However, this is not limitative.

In an environment without food, the nematodes search for food, that is, exhibit so-called searching behaviors. That is, the nematodes do not remain in a current position, but move over a wide range in the environment. Generally, as compared to an adult nematode of younger age of days, an adult nematode of older age of days tends to exhibit a low motility and a shorter total migration length per unit time. Thus, evaluation of the motility is useful as one indicator for aging analysis. In addition, different kinds of mutants exhibit different motilities. Thus, the evaluation of the motility is useful also for the purpose of checking a phenotype. The motility varies also depending on the presence or absence of an external stimulation and the various environmental conditions. Thus, the evaluation of the motility is useful also for the purpose of evaluation of a stimulus response. However, it is not easy to measure a total migration length of each nematode individual per unit time. An exemplary evaluation test that can be carried out in a simple manner and that can be used as an alternative to the evaluation of the motility from the total migration length per unit time will be described here. Specifically, the exemplary evaluation test estimates the motility of the nematodes from the number of nematodes caught in a fall-in cavity located at a position sufficiently separated from the initial position relative to the body lengths of the nematodes.

The following will describe the motility evaluation test of the nematodes. This test uses the nematode trap plate, an example of which is shown in (e) and (f) of FIG. 2, having the fall-in cavity formed in the outer periphery of the solid medium. Approximately 100 nematodes are released into the center of the solid medium, and the number of nematodes caught in the fall-in cavity is counted after a certain duration has elapsed. The duration to conduct the test may be freely set depending on the size of the nematode trap plate and the purpose of the test, generally within a range from several tens of minutes to approximately ten hours or more. The methods described in the above-described method for evaluating the stress degree of the nematodes may be applied to the liquid filled in the fall-in cavity, the method for capturing an image of the nematodes, and the method for collecting the nematodes in the present embodiment. The conditions, methods, and/or the like described for the above-described method for evaluating a response of the nematodes to the test subject may be applied to various other conditions such as the temperature condition and the light condition in the present embodiment.

The motility of the nematodes can be evaluated, for example, based on a value obtained by dividing the number of nematodes caught in the fall-in cavity by the total number of nematodes that are to be tested. Alternatively, it is possible to use, as an index for the evaluation, a duration taken until a certain percentage of individuals among the nematodes to be tested are caught in the fall-in cavity.

[Nematode Trap Test Kit]

A nematode trap test kit in accordance with the present embodiment includes at least the above-described nematode trap plate and the above-described cover for maintaining the environment on the solid medium of the nematode trap plate so as to be fixed. Typically, the nematode trap test kit corresponds to the nematode trap plate to which the cover is attached. Preferable aspects of the nematode trap plate and the cover are as described above. Preferably, the nematode trap test kit has a stacking mechanism with which plural nematode trap plates to which covers are attached can be stacked and stored.

A nematode trap test kit in accordance with another aspect of the present embodiment includes nematodes to be used in the test. In the nematode trap test kit including the nematodes, the nematodes can be provided in a container separated from a nematode trap plate and a cover.

There is no particular limitation on the numbers of nematode trap plates, covers, and nematodes in a single nematode trap test kit.

[Nematode Trap Plate Production Kit]

A nematode trap plate production kit in accordance with the present embodiment includes at least a container having the above-described through hole formed therein and a tube to be inserted into the through hole. Typically, the container is a raised-bottom container having a plurality of through holes into which tubes are to be inserted. Preferable aspects of the container and the tube are as described above.

In another aspect of the nematode trap plate production kit in accordance with the present embodiment, a material of a solid medium is included. In the nematode trap plate production kit including the material of the solid medium, the material can be provided in a container separated from the container and the tube.

Similarly to the above-described nematode trap test kit, the nematode trap plate production kit in accordance with another aspect of the present embodiment further includes a cover for maintaining the environment on the solid medium of the produced nematode trap plates so as to be fixed or nematodes to be used in the test.

There is no particular limitation on the numbers of containers, tubes, and materials of the solid medium as well as the kind and the number of nematodes in a single nematode trap plate production kit.

[Cancer Screening Test Method]

As described above, there has been known a cancer screening test that uses urine and nematodes (C. elegans) as a biosensor. In the generally-known cancer screening test that uses the urine and the nematodes, the nematode trap plate in accordance with the present embodiment can suitably be used. Specifically, in the olfaction test described in [Method for evaluating response of nematodes to test subject] above, the generally-known cancer screening test can be conducted by using, as a test subject, urine obtained from a testee. Here, the testee means mammal, specifically, human or mammal that is not human. Specific examples of the mammal that is not the human encompass pet animals such as canine, feline, rabbit, hamster, and ferret and industrial animals such as monkey, horse, cattle, pig, sheep, mouse, rat, and marmot. Particularly, the recent zoos have an additional important mission of maintaining and breeding animals of endangered species. Thus, a mammal individual of an endangered species raised in the zoo is quite important for maintaining the species. Therefore, health care for the individual is important. The urine of the individual is of course encompassed in the test subject to be used in the health care. Note that, typically, the animals of endangered species refer to animals belonging to Endangered Class IA for species that are facing an extremely high risk of extinction, Endangered Class IB for species that are facing a high risk of extinction, and Endangered Class II for species with an increasing risk of extinction in the International Union for Conservation of Nature (IUCN) Red List of species of wildlife that are facing a risk of extinction.

The type of cancer to be screened is not limited to any particular one, provided that an animal individual having the cancer can produce urine that can induce a nematode's behaviors of being attracted to the urine or a nematode's behavior of avoiding the urine. Examples of the cancer encompass malignant tumors. Specifically, the cancer to be screened can be a solid cancer, particularly, a carcinoma (an epithelial malignant tumor) or a sarcoma (a nonepithelial malignant tumor). The cancer to be screened may be a primary cancer or a metastatic tumor. Specific examples of the cancer can encompass a prostate cancer, a lingual cancer, a laryngeal cancer, an esophageal cancer, a carotid body tumor, a stomach cancer, a lung cancer, pulmonary neoplasia, a breast cancer, thymoma, a pericardial tumor, a colon cancer, a liver cancer, a hepatocellular carcinoma, a kidney cancer, a bile duct cancer, a pancreatic cancer, a bladder cancer, a testis cancer, a cervical cancer, an ovarian cancer, a squamous cancer, a malignant melanoma, osteosarcoma, a joint tumor, and a brain tumor.

After the test is ended, based on the number of nematodes caught in the fall-in cavity, it is determined whether or not there is a possibility that the testee has cancer. For example, a chemotaxis index (C.I.) is calculated. Then, if the chemotaxis index thus obtained is not less than or not more than a threshold preliminarily set for each concentration of a sample, it is determined that there is a possibility that the testee has cancer. Meanwhile, if the chemotaxis index thus obtained is below or above the threshold, it is possible to determine that the possibility that the testee has cancer is low. Typically, in a case where a sample to be used is a sample having a quite high concentration (e.g., undiluted urine having been obtained), determination that the chemotaxis index is equal to or higher than the preliminarily set threshold can be used as a criterion for determining that there is a possibility that the testee has cancer. Meanwhile, in a case where a sample to be used is a sample having a quite low concentration (e.g., diluted urine prepared by diluting undiluted urine at a quite low concentration), determination that the chemotaxis index is equal to or lower than the preliminarily set threshold can be used as a criterion for determining that there is a possibility that the testee has cancer.

With the cancer screening test method in accordance with the embodiment of the present invention, in tests and researches involving use of a laboratory animal such as monkey, mouse, rat, or marmot, determination of whether or not the laboratory animal has developed cancer can be made regularly in a simple manner from a sample that is a body fluid such as urine, without an autopsy or an image diagnosis. Avoidance of an autopsy, which can be carried out only once, means that it is possible to analyze the disease state of a single individual over time. This also leads to a reduction in the number of laboratory animal individuals to be sacrificed. In addition, this makes it possible to find development of a small cancer that cannot be found by an image diagnosis and eliminates the need for anesthesia generally carried out to, e.g., suppress movement of the laboratory animal in the image diagnosis, thereby making it possible to avoid unexpected death or damage of the laboratory animal that might otherwise be caused by administration of an excess amount of anesthetic. Moreover, the cancer screening test method in accordance with the embodiment of the present invention is applicable to, e.g., evaluation of the risk of carcinogenesis by the drug carried out with use of a laboratory animal and evaluation of the anticancer effect of the drug carried out with use of mice bearing cancer in drug toxicity tests for medical and pharmaceutical products and the like. Consequently, the evaluation in the drug toxicity test can be carried out regularly in a simple manner. Furthermore, the cancer screening test method in accordance with the embodiment of the present invention is applicable to determination of whether or not a laboratory animal has developed cancer in researches for the risk of carcinogenesis focusing on various factors such as radiation exposure and aging.

The cancer screening test method in accordance with the present embodiment, in which the nematode trap plate is used, can carry out a screening test in a simple manner with a high evaluation accuracy.

The present embodiment has dealt with the cancer screening test. In addition, it is possible to determine, from a certain body fluid, the physiological condition of the testee in the same manner as in the above-described cancer screening test, in a case where the body fluid of the testee being in a different condition from that of a healthy individual induces a nematode's behavior of being attracted to the body fluid or a nematode's behavior of avoiding the body fluid. Thus, in an aspect of the method for evaluating a response of nematodes, the test subject can be a body fluid of mammal. The body fluid is not limited to any specific kind. Preferably, the body fluid is a body fluid that can be easily obtained from mammal to be tested, examples of which encompass urine, sweat, tears, blood, saliva, and mucus.

As described above, in the nematode trap plate in accordance with the present embodiment and the response evaluation carried out with use of the nematode trap plate, nematodes are trapped by making use of the phenomenon that the nematodes having fallen into a fall-in cavity as a result of being attracted to a test subject/temperature or avoiding the test subject/temperature cannot move back onto the solid medium. Thus, regardless of the lapsed duration, it is possible to accurately evaluate the number of nematodes caught in the fall-in cavity as a result of being attracted to the subject or avoiding the subject. In addition, the number of nematodes having been caught may be increased as time passes. However, the number of nematodes having been caught would not be reduced even as time passes. Thus, the timing to carry out the evaluation of the response may be set substantially within a range from several minutes to one day after start of the test, in consideration of a duration taken until the test solution and the control liquid are evaporated. This timing does not greatly affect the accuracy in evaluation. Thus, the burden on a person who conducts the test is significantly improved. Moreover, it is not necessary to use an anesthetic that the conventional chemotaxis evaluation methods use to keep the nematodes within an area into which the nematodes have been attracted, thereby making it possible to purely evaluate a response of attraction to or avoidance of a certain substance and a certain temperature. Furthermore, in accordance with the method in which the test solution is poured into the fall-in cavity, even if a test subject of an olfaction test is a substance having a taste, it is possible to examine merely a response to an odor independently of the taste. This means that, even in a case of using a sample generally having a high sugar content such as urine of a testee having diabetes, it is not necessary to consider the effect of the taste of the sample on an olfaction response of the nematodes. In addition, since the preparation for the test is completed merely by forming a fall-in cavity and filling the fall-in cavity with a liquid, it is possible to significantly increase the efficiency of the test itself.

Moreover, image-capturing or observation needs to be carried out only on the inside of the fall-in cavity or the inside of the tube or the like into which the trapped nematodes are collected, not on the whole of the solid medium, and the number of nematodes needs to be measured only in the fall-in cavity or the tube or the like. Thus, the range to be subjected to image-capturing or observation is limited to a small range. This makes it possible to evaluate the response in a simple manner, thereby significantly improving the efficiency.

Furthermore, in a case of using transgenic nematodes modified to emit fluorescence, it is possible to calculate, from a fluorescence intensity, the number of nematodes inside the fall-in cavity or the tube or the like by capturing a fluorescent image of the inside of the fall-in cavity into which the nematodes have been caught or the inside of the tube or the like into which the trapped nematodes have been collected. Carrying out calibration of a relation between a fluorescence intensity and the number of individuals in advance makes it possible to accurately calculate the number of nematodes caught in the fall-in cavity, thereby eliminating the need to manually count the number of nematodes in an image captured in each test. This significantly reduce the duration taken until the result is obtained, thereby significantly saving the effort in testing.

The above-described nematode trap plate in accordance with the present embodiment and techniques that uses the nematode trap plate can be used in various industrial fields such as medical science and medical service, life science, veterinary science and veterinary service, agriculture and forestry, food, quantum science, and environmental field. Particularly, in the fields of medical science and medical service as well as veterinary science and veterinary service, the present invention is remarkably high in use value, since a non-invasive cancer screening test involving use of urine can be established on the basis of the present invention. The non-invasive screening method involving use of the nematode trap plate in accordance with the present embodiment may be used to establish early detection and diagnosis of cancer and monitoring of the effect of therapy on cancer in mammals such as human. It is expected that this may bring about innovation in preventive medical service, preventive veterinary service, and follow-up care of cancer therapy. In the fields of food, environmental science, and the like, application of the technique of the present embodiment to searching for a toxic substance for an organism is expected, while focusing on the nematode's behavior of avoidance of a test subject. In addition, the technique of the present embodiment is applicable also to nematode screening for electing a nematode suitable for each test.

The following will provide Examples to more specifically describe embodiments of the present invention. As a matter of course, the present invention is not limited to Examples provided below, but details of the present invention can be realized in various manners. Further, the present invention is not limited to the embodiments described above, and it may be varied in various ways within the scope of the appended Claims. Thus, an embodiment achieved by combining technical means varied appropriately within the scope of the appended claims will be included by the technical scope of the present invention. In addition, the contents of all the literatures referred herein are incorporated herein by reference in their entirety.

### EXAMPLES

In the Examples shown below, tests were conducted by using C. elegans as the nematodes, unless otherwise stated. In each of the Examples, a commercially-available circular plastic dish was used as the container of the nematode trap plate. The size of the plastic dish, the capacity (volume) of a solid medium mainly made of an agar, and the number of fall-in cavities to be formed were set as appropriate in consideration of the purpose of the test, the volume of a sample obtained, and the like. In each case, the fall-in cavities were formed in the shape of a cylinder having a diameter of approximately 5 mm. Table 1 shows details of the plates that were used. The following description will be made by using the names of the plates shown in Table 1 (plate A1, plate A2, plate B1, plate B2, plate B3, plate B4, and plate B5).

TABLE 1

| | | | | | Diameter | |
|---|---|---|---|---|---|---|
| | Inner | | | Number | (mm) of | |
| | diameter | | Volume | of fall-in | each | |
| | (cm) of | Bottom | (mL) of | cavities | fall-in | |
| | bottom | area | solid | 2a and | cavity 2a | Example |
| Name | surface | (cm²) | medium | 2b | and 2b | (s) |
| Plate | Approx. | Approx. | 10.0 | Two | 5.0 | 1, 15 |
| A1 | 8.4 | 55.0 | | for each | | |
| Plate | Approx. | Approx. | 20.0 | Two | 5.0 | 2 |
| A2 | 8.4 | 55.0 | | for each | | |
| Plate | Approx. | Approx. | 10.0 | One | 5.0 | 3, 4, 5, 9 |
| B1 | 5.2 | 21.0 | | for each | | |
| Plate | Approx. | Approx. | 10.0 | Two | 5.0 | 6, 7, 8, |
| B2 | 5.2 | 21.0 | | for each | | 9, 10 |
| Plate | Approx. | Approx. | 3.0 | Two | 5.0 | 8, 9, 11 |
| B3 | 5.2 | 21.0 | | for each | | |
| Plate | Approx. | Approx. | 3.0 | One | 5.0 | 12, 13, 14 |
| B4 | 5.2 | 21.0 | | for each | | |
| Plate | Approx. | Approx. | 3.0 | One | 5.0 | 16 |
| B5 | 5.2 | 21.0 | | (only | | |
| | | | | one 2a) | | |

### Example 1

### Test 1 for Evaluating Response of Nematodes to Volatile Substance

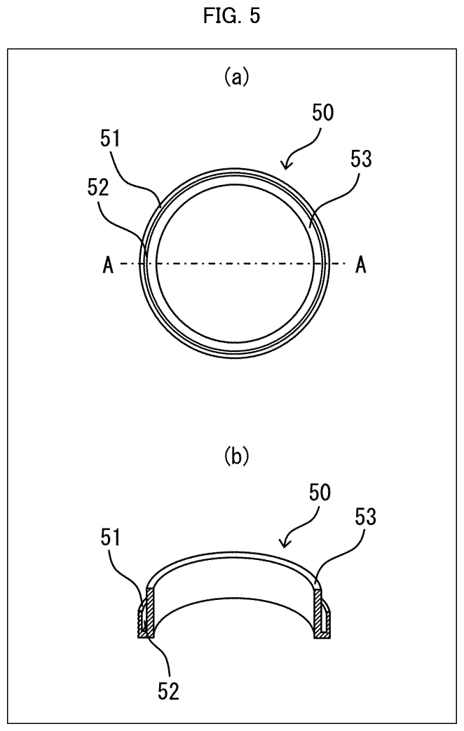

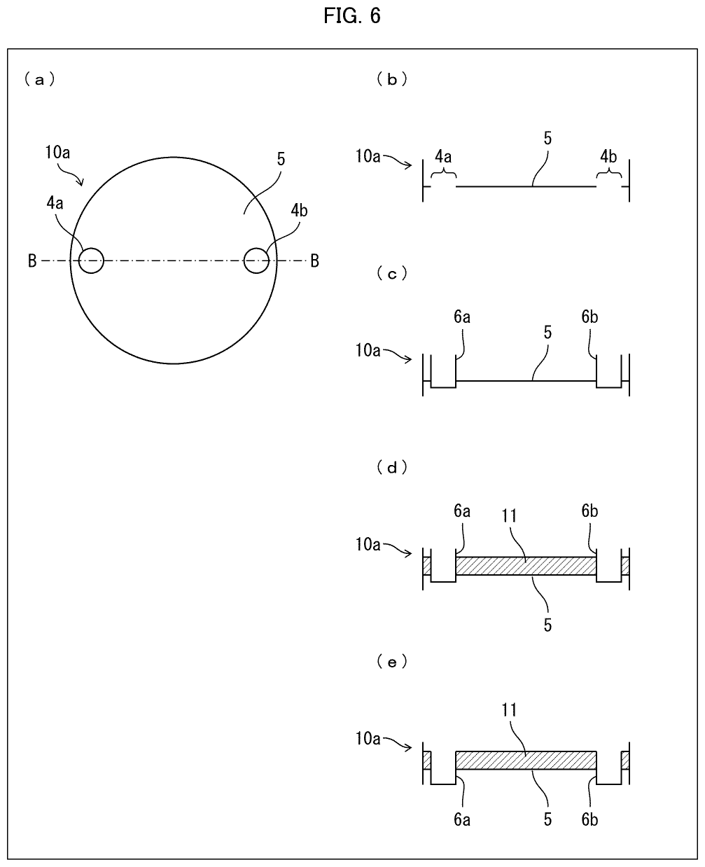

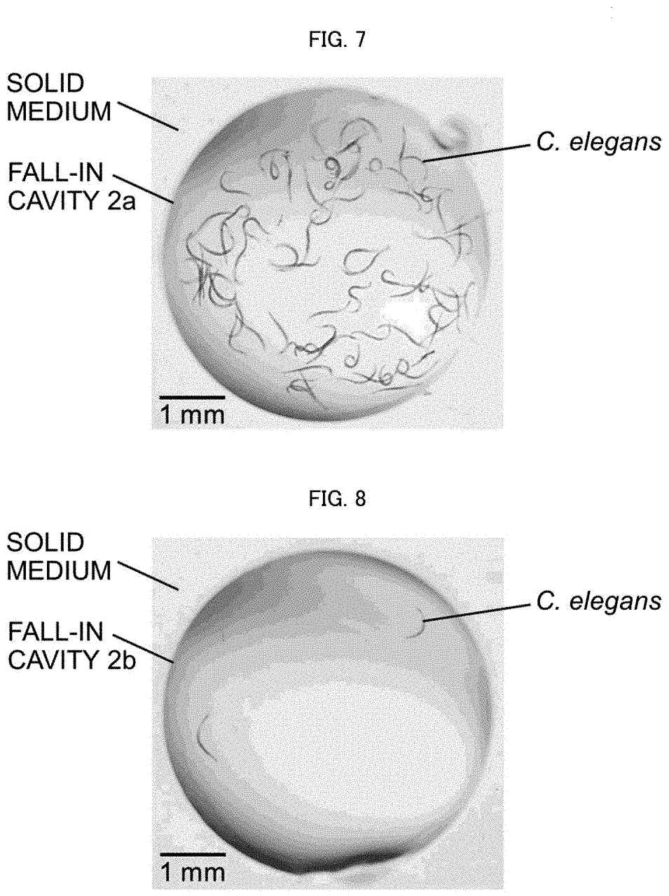

A test for evaluating a response of nematodes to a volatile substance was conducted with use of diacetyl (with a molarity of 11.5 M at 15° C.), which is a kind of volatile substance, and diluted solutions prepared by diluting diacetyl at five different concentrations ranging from 10-fold to 100000-fold. First, a plate A1 was produced that included (a) two fall-in cavities 2a formed in a left part of a solid medium so as to be arranged side by side along the outer periphery of the solid medium and (b) two fall-in cavities 2b formed in control positions in a right part of the solid medium in a similar manner to the fall-in cavities 2a. The left fall-in cavities 2a were filled with a liquid to be tested (it may sometimes be referred to as a “test solution”), whereas the right fall-in cavities 2b were filled with a buffer solution as a control. As described above, the test solution used in Example 1 was an undiluted solution of diacetyl, which is a kind of volatile substance, or a diluted solution of diacetyl. Note that the dilution was conducted with use of the buffer solution, which was used as a control. Next, approximately 150 nematodes having been washed in advance with the buffer solution were released onto the center of the plate, and the plate was covered with a cover and sealed with tape. Then, the plate was left at least for an hour under a light-shielded environment. Thereafter, images of the fall-in cavities 2a and 2b were captured under a microscope. Based on the images showing the result of the test, examples of which are shown in FIGS. 7 and 8, the numbers of nematodes caught in the fall-in cavities 2a and 2b were counted.

(Result)

Assuming that the total number of nematodes caught in the two left fall-in cavities 2a filled with the test solution was Ni and the total number of nematodes caught in the two right fall-in cavities 2b filled with the control buffer solution was N₂, a value obtained according to the following formula was defined as a chemotaxis index (C.I.). Then, the response was evaluated.

Chemotaxis index (C.I.)=(N₁−N₂)/(N₁+N₂).

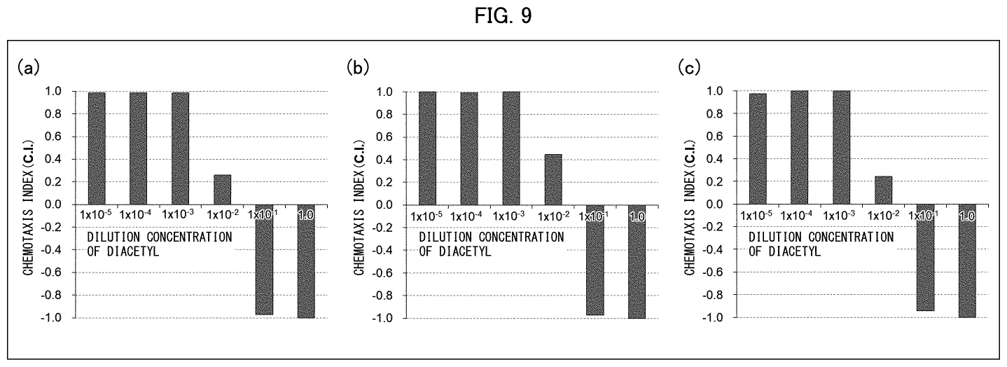

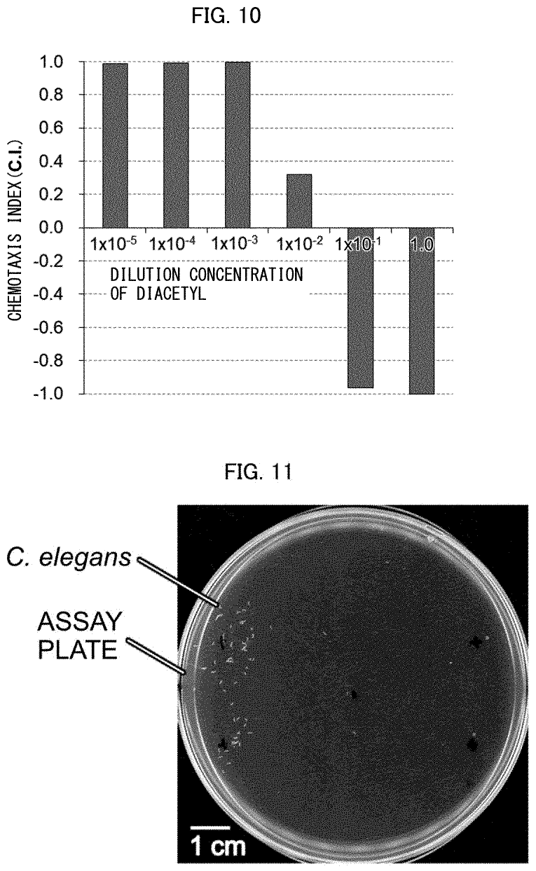

A positive value means attraction to the left fall-in cavities 2a, that is, to diacetyl, whereas a negative value means escaping from the fall-in cavities 2a. Note that tests were conducted with use of an undiluted solution of diacetyl (with a molarity of 11.5 M at 15° C.) and diluted solutions prepared by diluting diacetyl at five different concentrations, that is, the tests were conducted on test solutions of six different concentrations. (a) to (c) of FIG. 9 show the C.I. values obtained in three times of tests conducted independently. FIG. 10 shows mean values thereof.

(a) to (c) of FIG. 9 show the following. That is, in all of the three times of tests conducted, negative values, which means escaping of the nematodes from diacetyl, were obtained at two highest concentrations among the six dilution concentrations. Meanwhile, at four other dilution concentrations, positive values were obtained. This indicates that the nematodes were attracted to diacetyl. The results of the tests shown in (a) to (c) of FIG. 9 and the mean values of the results of the three times of tests shown in FIG. 10 exhibited a similar tendency. This reveals that the present invention can quantify, with a high sensitivity and a high reproducibility, changes in response of nematodes caused by the concentration of the substance.

### Comparative Example 1

### Test for Evaluating Response of Nematodes to Volatile Substance in Accordance with Conventional Method

As a comparative example, evaluation of a response of nematodes was conducted in accordance with a conventional chemotaxis evaluation method (e.g., Non-Patent Literature 1). A test in accordance with the conventional method was conducted with use of a plate (hereinafter, such a plate may sometimes be referred to as an “assay plate”) identical to the plate A1 used in the test of Example 1, except that the assay plate did not have the fall-in cavities 2a and 2b. Similarly to the test in Example 1, an undiluted solution of diacetyl and diluted solutions prepared by diluting diacetyl at five different concentrations were used as test solutions. One drop of the test solution was dripped on each of two positions (test solution spots) on a left half of a circle with a radius of 3 cm about the center of the assay plate. Meanwhile, the buffer solution serving as the control was dripped on each of positions (control spots) that were on a right half of the circle with a radius of 3 cm about the center of the assay plate and that were arranged point symmetrically. Next, one drop of 0.5 M sodium azide, which is a kind of anesthetic, was dripped on each of the four spots. The assay plate was covered with a cover and left until the dripped liquids were dried or the dripped liquids were soaked into the solid medium filled in the assay plate. Thereafter, approximately 100 nematodes having been washed in advance with the buffer solution were released onto the center of the assay plate, and the assay plate was covered with the cover and sealed with tape. Then, the plate was left at least for an hour under a light-shielded environment. An image of the entire assay plate was captured through the bottom surface. Based on the captured image of the entire assay plate, an example of which is shown in FIG. 11, the number of nematodes fixed under anesthesia was counted in each of test solution areas extending concentrically from the two test solution spots on the left part and control areas extending concentrically from the two control spots on the right part. The settings of the areas were selected according to the conventionally-known nematode chemotaxis evaluation method.

(Result)

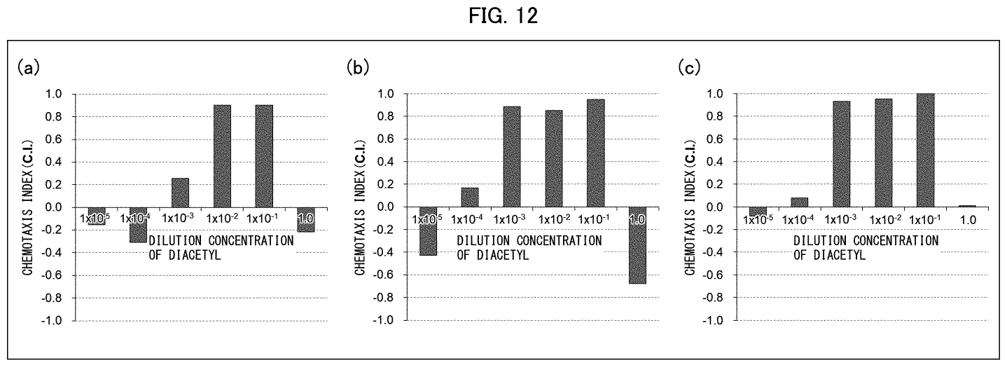

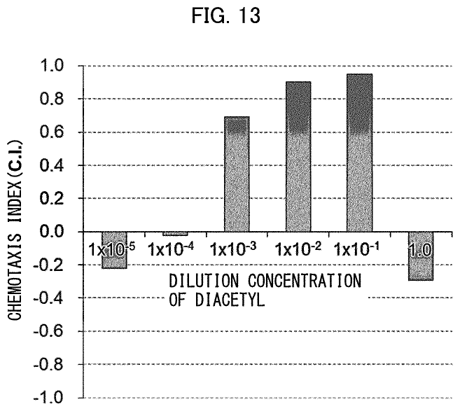

The response of the nematodes was evaluated with use of the chemotaxis index (C.I.), which was used also in Example 1, assuming that the number of nematodes in the test solution areas was N₁ and the number of nematodes in the control areas was N₂. A positive value means attraction to the test solution areas, that is, to diacetyl, whereas a negative value means escaping from the test solution areas. (a) to (c) of FIG. 12 show the C.I. values obtained in three times of tests conducted independently. FIG. 13 shows mean values thereof.

As shown in (a) to (c) of FIG. 12, with the conventional method, different responses were observed for the different doses in the three times of tests conducted. Thus, a large variation was recognized. Particularly, in the cases where the undiluted solution and the test solution with a large dilution factor were used, a positive value was obtained in one case, and a negative value was obtained in another case. Thus, instability was observed. The mean values of C.I. in the three times of tests shown in FIG. 13 do not reflect well the results of the individual tests. This reveals that the conventional method, which has a poor reproducibility, involves a big problem in terms of quantitativeness.

### Example 2

### Test 2 for Evaluating Response of Nematodes to Volatile Substance

Example 2 was conducted for the purpose of examining whether or not a difference in thickness of the plate changes the nematode response. Specifically, a plate A2 was produced that was identical to the plate A1 of Example 1 in terms of the size and shape of the plate and the shapes of the fall-in cavities, except that the capacity (volume) of a solid medium of the plate 2 was substantially twice as great as that of the plate A1. That is, in the plate A2, the capacity (volume) of a liquid that can be filled in fall-in cavities 2a and 2b was substantially twice as great as that in the plate used in Example 1. The left fall-in cavities 2a were filled with a test solution, whereas the right fall-in cavities 2b were filled with a buffer solution as a control. The test solution used in Example 2 was a diluted solution of diacetyl, which is a kind of volatile substance, and the dilution was conducted with use of the buffer solution, which was used as a control. The left fall-in cavities 2a were filled with a test solution, whereas the right fall-in cavities 2b were filled with a buffer solution as a control. Next, approximately 150 nematodes having been washed in advance with the buffer solution were released onto the center of the plate, and the plate was covered with a cover and sealed with tape. Then, the plate was left at least for an hour under a light-shielded environment. Thereafter, images of the fall-in cavities 2a and 2b were captured under a microscope. Based on the images, the numbers of nematodes caught in the fall-in cavities 2a and 2b were counted.

(Result)

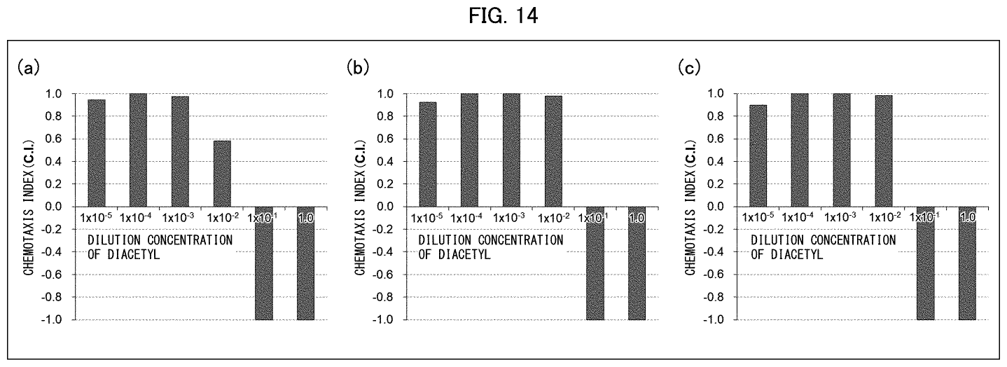

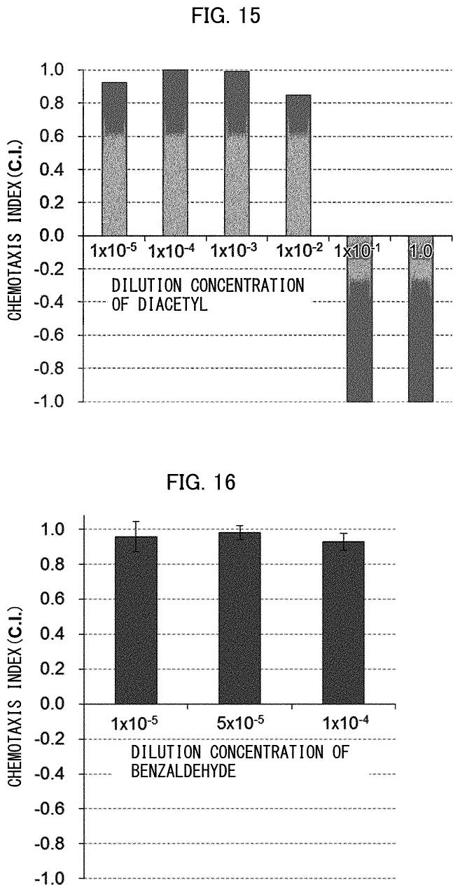

The chemotaxis index (C.I.) was obtained in a similar manner to Example 1, and the response of the nematodes was evaluated. A positive value means attraction to the left fall-in cavities 2a, that is, to diacetyl, whereas a negative value means escaping from the fall-in cavities 2a. Note that the tests were conducted on test solutions of six different concentrations, specifically, an undiluted solution of diacetyl (with a molarity of 11.5 M at 15° C.) and diluted solutions prepared by diluting the undiluted solution at five different concentrations ranging from 10-fold to 100000-fold. (a) to (c) of FIG. 14 show the C.I. values obtained in three times of tests conducted independently. FIG. 15 shows mean values thereof.

As shown in FIGS. 14 and 15, at all of the six different dilution concentrations having been tested, the same results as in Example 1 conducted with use of the plate A1, in which the depths of the fall-in cavities 2a and 2b and the thickness of the medium were approximately half of those in Example 2, were obtained. This reveals that a difference in thickness of the plate, that is, a difference in depth of the fall-in cavities does not greatly affect the evaluation of the response to the volatile substance.

### Example 3

### Test 3 for Evaluating Response of Nematodes to Volatile Substance

A plate B1, such as the one shown in FIG. 1, was produced. Specifically, the plate B1 was produced to have a diameter approximately two thirds of that of the plate A1 used in Example 1, one fall-in cavity 2a in a left part of a solid medium, and one fall-in cavity 2b at a control position in a right part of the solid medium. The left fall-in cavity 2a was filled with a test solution, whereas the right fall-in cavity 2b was filled with ultrapure water as a control. The test solution used in Example 3 was a diluted solution of benzaldehyde. Next, approximately 100 nematodes having been washed in advance with a buffer solution were released onto the center of the plate, and the plate was covered with a cover and sealed with tape. Then, the plate was left for an hour under a light-shielded environment. Thereafter, images of the fall-in cavities 2a and 2b were captured under a microscope. Based on the images, the numbers of nematodes caught in the fall-in cavities 2a and 2b were counted.

(Result)

Assuming that the number of nematodes caught in the left fall-in cavity 2a filled with the test solution was N₁ and the number of nematodes caught in the right fall-in cavity 2b used as the control was N₂, C.I. was obtained in a similar manner to Example 1. Then, the response of the nematodes was evaluated. A positive value means attraction to the left fall-in cavity 2a, that is, to benzaldehyde, whereas a negative value means escaping from the fall-in cavity 2a. Note that the tests were conducted on test solutions of three different concentrations, specifically, diluted solutions prepared by diluting an undiluted solution of benzaldehyde (with a molarity of 9.8 M at 15° C.) at three different concentrations ranging from 10000-fold to 100000-fold. FIG. 16 shows mean values of C.I. obtained in four times of tests conducted independently. The error bars in FIG. 16 each indicate a standard deviation.

As shown in FIG. 16, at each of the three different dilution concentrations having been tested, C.I. exhibited a value of not less than 0.9, which is close to the maximum value of 1.0. This shows that the nematodes were attracted to benzaldehyde. This result exhibits a similar tendency to that of the result of the chemotaxis test conducted according to the conventional method.

### Example 4

### Test for Evaluating Response of Nematodes to Urine of Patient with Prostate Cancer

A plate B1 identical to that produced in Example 3 was produced. A fall-in cavity 2a in a left part of a solid medium was filled with a test solution, whereas a fall-in cavity 2b in a right part of the solid medium was filled with a physiological saline solution as a control. A test solution used in Example 4 was a solution prepared by diluting, with a physiological saline solution, a mixture solution of urine samples obtained from 12 patients with a prostate cancer who had not taken a radiation therapy or a therapy with a drug yet. Next, approximately 100 nematodes having been washed in advance with a buffer solution were released onto the center of the plate. The plate was covered with a cover and sealed with tape. Then, the plate was left for an hour under a light-shielded environment. Thereafter, images of the fall-in cavities 2a and 2b were captured under a microscope, and the numbers of nematodes caught in the fall-in cavities 2a and 2b were counted based on the images.

(Result)

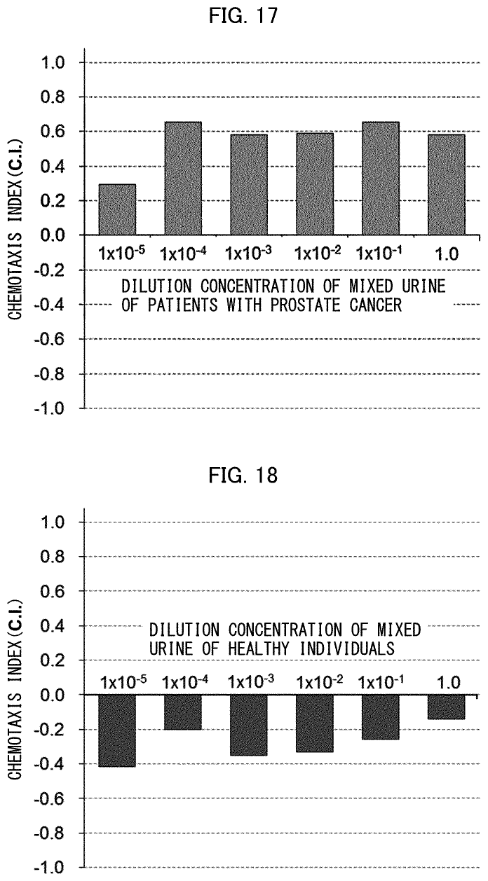

The chemotaxis index (C.I.) was obtained in a similar manner to Example 3, and the response of the nematodes was evaluated. A positive value means attraction to the left fall-in cavity 2a, that is, to urine of the patients with a prostate cancer, whereas a negative value means escaping from the fall-in cavity 2a. Note that the tests were conducted on test solutions of six different concentrations, specifically, the mixed urine and diluted solutions prepared by diluting the undiluted mixed urine at five different concentrations ranging from 10-fold to 100000-fold. FIG. 17 shows mean values of C.I. obtained in two times of tests conducted independently.

As shown in FIG. 17, at the six different dilution concentrations having been tested, positive C.I. values were obtained. This reveals that the nematodes were attracted to the urine of the patients with the prostate cancer. This result exhibits a similar tendency to that of the result of the chemotaxis test conducted according to the conventional method in which a urine sample obtained from each patient with a prostate cancer was solely used as a test subject. Although not detailed here, in another test involving use of urine samples collected from patients with a prostate cancer which urine samples were not mixed together but were individually diluted, it was confirmed that the nematodes tended to be attracted to the urine, similarly to the case involving use of the mixed urine.

### Example 5

### Test for Evaluating Response of Nematodes to Urine of Healthy Individual Having Never Developed Cancer

A plate B1 identical to that produced in Example 3 was produced. A fall-in cavity 2a in a left part of a solid medium was filled with a test solution, whereas a fall-in cavity 2b in a right part of the solid medium was filled with a physiological saline solution as a control. A test solution used in Example 4 was a solution prepared by diluting, with a physiological saline solution, a mixture solution of urine obtained from 12 healthy males having never developed cancer. Next, approximately 100 nematodes having been washed in advance with a buffer solution were released onto the center of the plate. The plate was covered with a cover and sealed with tape. Then, the plate was left for an hour under a light-shielded environment. Thereafter, images of the fall-in cavities 2a and 2b were captured under a microscope, and the numbers of nematodes caught in the fall-in cavities 2a and 2b were counted based on the images.

(Result)

The chemotaxis index (C.I.) was obtained in a similar manner to Example 3, and the response of the nematodes was evaluated. A positive value means attraction to the left fall-in cavity 2a, that is, to urine of a healthy individual, whereas a negative value means escaping from the fall-in cavity 2a. Note that the test was conducted on test solutions of six different concentrations, specifically, mixed urine and diluted solutions prepared by diluting the undiluted mixed urine at five different concentrations ranging from 10-fold to 100000-fold. FIG. 18 shows mean values of C.I. obtained in two times of tests conducted independently.

As shown in FIG. 18, at the six different dilution concentrations having been tested, negative C.I. values were obtained. This reveals that the nematodes escaped from the urine of the healthy individual having never developed cancer. This result shows an avoidance response, which is opposite to the attraction response to the urine of the cancer patient observed in Example 4. Thus, this result exhibits a similar tendency to that observed in the chemotaxis test conducted according to the conventional method in which a urine sample obtained from each healthy individual was solely used as a test subject. Although not detailed here, in another test involving use of urine samples collected from health individuals which urine samples were not mixed together but were individually, it was confirmed that the nematodes tended to avoid the urine, similarly to the case involving use of the mixed urine.

It should be noted that the results of Examples 4 and 5 show that the test for evaluating a response of the nematodes can be conducted either in a case where a urine sample obtained from each subject is solely used as a test subject or a case where a mixture of urine samples obtained from plural subjects is used as a test subject.

In view of the results of Examples 4 and 5, it is possible to examine the possibility that the subject has cancer in the following manner. That is, for example, zero can be set as a threshold under the test conditions of Examples 4 and 5. Then, in a case where a positive chemotaxis index (C.I.) is obtained, the subject can be determined as cancer positive. Meanwhile, in a case where a negative chemotaxis index is obtained, the subject can be determined as cancer negative. In addition, also in the tests for evaluating a response to urine conducted in Example 6 and its subsequent Examples, it is possible to set a threshold as appropriate to examine the possibility that the subject has cancer. Since the determination condition varies depending on the kind and capacity (volume) of the sample, the test conditions, and/or the like, a threshold for a cancer screening test is to be set according to the content and condition of the test.

### Example 6

### Test for Evaluating Response of Nematodes to Urine of Male Canine Having Never Developed Cancer

A plate B2 was produced that was identical to the plate of Example 3 in terms of the size and shape of the plate and the sizes and shapes of fall-in cavities, except that the number of fall-in cavities of the plate B2 was four. That is, in the plate B2, two fall-in cavities 2a were provided in a left part of a solid medium, and two fall-in cavities 2b were provided at control positions in a right part of the solid medium. The left fall-in cavities 2a were filled with a test solution, whereas the right fall-in cavities 2b were filled with a physiological saline solution as a control. The test solution used in Example 6 was urine obtained from a male canine having never developed cancer. Next, approximately 150 nematodes having been washed in advance with a buffer solution were released onto the center of the plate. The plate was covered with a cover and sealed with tape. Then, the plate was left for an hour under a light-shielded environment. Thereafter, images of the fall-in cavities 2a and 2b were captured under a microscope, and the numbers of nematodes caught in the fall-in cavities 2a and 2b were counted based on the images.

(Result)

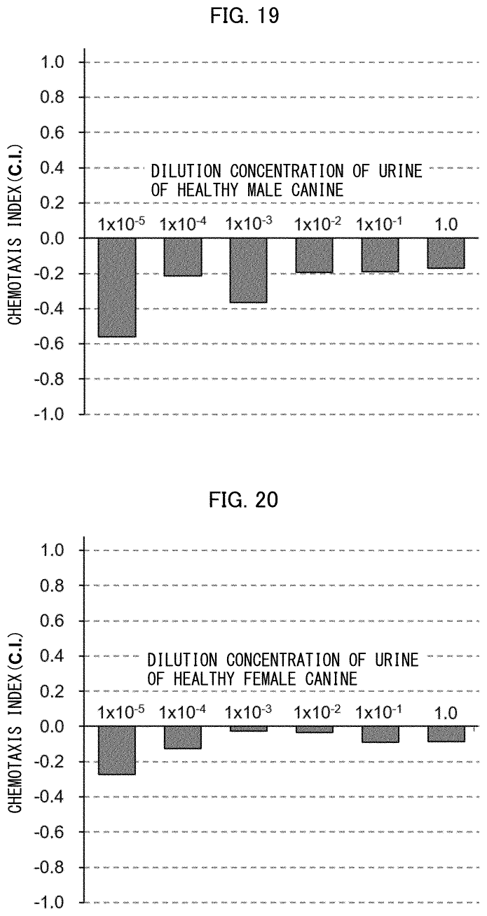

The chemotaxis index (C.I.) was obtained in a similar manner to Example 1, and the response of the nematodes was evaluated. A positive value means attraction to the left fall-in cavities 2a, that is, to the odor of the urine of a healthy male canine, whereas a negative value means escaping from the fall-in cavities 2a. Note that the tests were conducted on test solutions of six different concentrations, specifically, undiluted urine and diluted solutions prepared by diluting the undiluted urine at five different concentrations ranging from 10-fold to 100000-fold. FIG. 19 shows the result.

As shown in FIG. 19, at the six different dilution concentrations having been tested, negative C.I. values were obtained. This reveals that the nematodes escaped from the urine of the male canine having never developed cancer. This result shows an avoidance response, which is similar to the response observed in Example 3 with respect to the urine of the healthy individual having never developed cancer. From the above results, it was confirmed that, with the present invention, cancer screening can be conducted on a sample of a different kind of mammal in the same manner as that for a sample obtained from human. In addition, it is apparent from the result of Example 5 that the same test result will be obtained with use of either the plate having two fall-in cavities or the plate having four fall-in cavities.

[Example 7. Test for evaluating response of nematodes to urine of female canine having never developed cancer] A plate B2 identical to that produced in Example 6 was produced. Left fall-in cavities 2a were filled with a test solution, whereas right fall-in cavities 2b were filled with a physiological saline solution as a control. The test solution used in Example 7 was urine obtained from a female canine having never developed cancer. Next, approximately 150 nematodes having been washed in advance with a buffer solution were released onto the center of the plate. The plate was covered with a cover and sealed with tape. Then, the plate was left for an hour under a light-shielded environment. Thereafter, images of the fall-in cavities 2a and 2b were captured under a microscope, and the numbers of nematodes caught in the fall-in cavities 2a and 2b were counted based on the images.

(Result)

The chemotaxis index (C.I.) was obtained in a similar manner to Example 1, and the response of the nematodes was evaluated. A positive value means attraction to the left fall-in cavities 2a, that is, to the urine of a healthy female canine, whereas a negative value means escaping from the fall-in cavities 2a. Note that the tests were conducted on test solutions of six different concentrations, specifically, undiluted urine and diluted solutions prepared by diluting the undiluted urine at five different concentrations ranging from 10-fold to 100000-fold. FIG. 20 shows the result.

As shown in FIG. 20, at the six different dilution concentrations having been tested, negative C.I. values were obtained. This reveals that the nematodes escaped from the urine of the female canine having never developed cancer.

This result shows an avoidance response, which is similar to the response observed in Example 5 with respect to the urine of the healthy individual having never developed cancer and the response observed in Example 6 with respect to the urine of the healthy male canine having never been developed cancer. From the above results, it was confirmed that, with the present invention, cancer screening can be conducted on a sample of a different kind of mammal of any sex in the same manner as for a sample obtained from human.

### Example 8

### Test for Evaluating Response of Nematodes to Urine of Plural Canines with Cancer

A plate B2 identical to that produced in Example 6 was produced for most cases. In addition to this, a plate B3 was specially produced and used in a case where the capacity (volume) of a test solution obtained was small. The plate B3 was identical to the plate B2 in terms of the size and shape of the plate and the shapes of fall-in cavities, except that the capacity (volume) of a solid medium of the plate B3 was three tenths of that of the plate B2. Left fall-in cavities 2a were filled with a test solution, whereas right fall-in cavities 2b were filled with a physiological saline solution as a control. The test solution used in Example 8 was urine obtained from 11 canines with cancer. Next, approximately 150 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

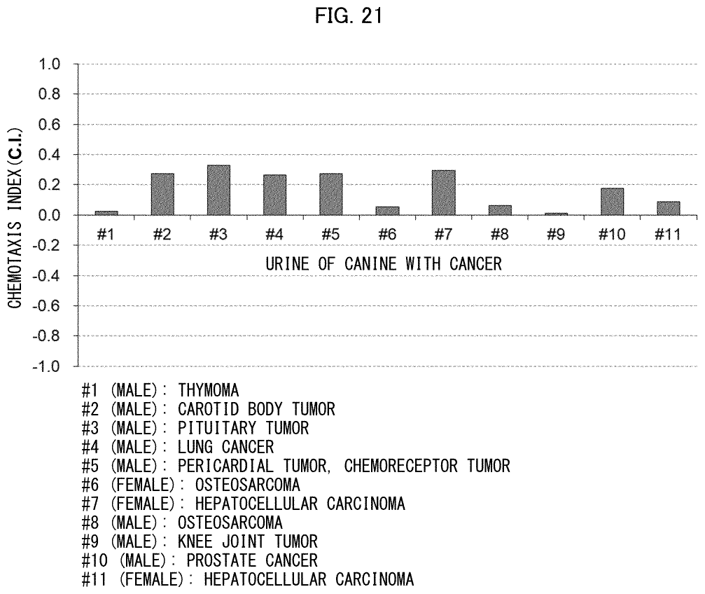

The chemotaxis index (C.I.) was obtained in a similar manner to Example 1, and the response of the nematodes was evaluated. A positive value means attraction to the left fall-in cavities 2a, that is, to the urine of the canine with cancer, whereas a negative value means escaping from the fall-in cavities 2a. Note that the tests on the urine samples of the 11 canines with cancer were conducted independently. The types of the cancers in the 11 canines confirmed through image diagnosis and/or the like were thymoma, a carotid body tumor, a pituitary tumor, a lung cancer, a combination of a pericardial tumor and a chemoreceptor tumor, osteosarcoma (two canines), a hepatocellular carcinoma (two canines), a knee joint tumor, and a prostate cancer. FIG. 21 shows mean values of C.I. obtained in three times of tests conducted independently with use of these urine samples.

As shown in FIG. 21, with all the tested urine samples of the 11 canines with cancer, positive C.I. values were obtained. This reveals that the nematodes were attracted to the urine of the canines with cancer. It is apparent from this result that the nematodes are attracted to the urine regardless of the type of the cancer. The 11 canines included male canines and female canines. That is, it was confirmed that, with the present invention, it is possible to detect various types of cancers in a canine of any sex.

[Example 9. Test for evaluating response of nematodes to urine of plural canines having never developed cancer]

A plate B2 identical to that produced in Example 6 was produced for most cases. In addition to this, a plate B1 or a plate B3 was produced and used in a case where the capacity (volume) of a test solution obtained was small. The plate B1 was identical to the plate B2 in terms of the size and shape of the plate and the sizes and shapes of fall-in cavities, except that the number of fall-in cavities of the plate B1 was two. The plate B3 was identical to the plate B2 in terms of the size and shape of the plate and the shapes of fall-in cavities, except that the capacity (volume) of a solid medium of the plate B3 was three tenths of that of the plate B2. A left fall-in cavity(ies) 2a was/were filled with a test solution, whereas a right fall-in cavity(ies) 2b was/were filled with a physiological saline solution as a control. The test solution used in Example 9 was urine obtained from six canines having never developed cancer. Next, approximately 150 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

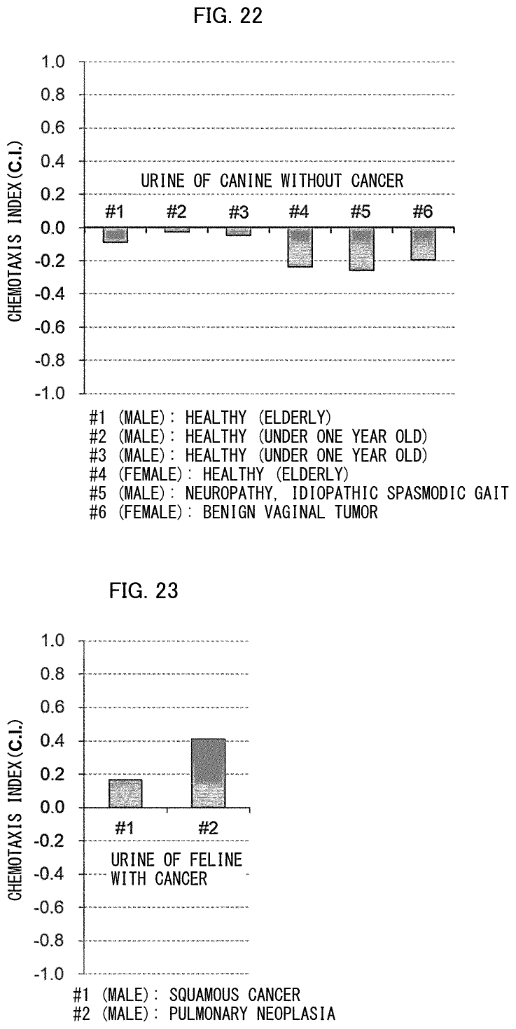

For the plate including the four fall-in cavities, a chemotaxis index (C.I.) was obtained in a similar manner to Example 1. For the plate including the two fall-in cavities, a chemotaxis index was obtained in a similar manner to Example 3. Then, the response of the nematodes was evaluated. A positive value means attraction to the left fall-in cavity(ies) 2a, that is, to the urine of the canine having never developed cancer, whereas a negative value means escaping from the fall-in cavity(ies) 2a. Note that the tests on the urine samples of the six canines having never been developed cancer were conducted independently. The six canines included two elderly healthy individuals, two healthy individuals under one year old, one individual with neuropathy and idiopathic spasmodic gait, and one individual with a benign vaginal tumor. FIG. 22 shows mean values of C.I. obtained in two or three times of tests conducted independently with use of these urine samples.

As shown in FIG. 22, with all the tested urine samples of the six canines having never developed cancer, negative C.I. values were obtained. This reveals that the nematodes escaped from the urine of the canines having never developed a cancer. From the above result, it was confirmed that, with the present invention, not only a healthy canine having never developed cancer but also a canine having neuropathy or a benign tumor would be rarely erroneously determined as a canine with cancer.

### Example 10

### Test for Evaluating Response of Nematodes to Urine of Plural Felines with Cancer

A plate B2 identical to that produced in Example 6 was produced. Left fall-in cavities 2a were filled with a test solution, whereas right fall-in cavities 2b were filled with a physiological saline solution as a control. The test solution used in Example 10 was urine obtained from two felines with cancer. Next, approximately 150 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

The chemotaxis index (C.I.) was obtained in a similar manner to Example 1, and the response of the nematodes was evaluated. A positive value means attraction to the left fall-in cavities 2a, that is, to the urine of the feline with cancer, whereas a negative value means escaping from the fall-in cavities 2a. Note that the tests on the urine samples of the two felines with cancer were conducted independently. The types of the cancers in the two felines confirmed through image diagnosis and/or the like were a squamous cancer and a pulmonary neoplasia. FIG. 23 shows mean values of C.I. obtained in three times of tests conducted independently with use of these urine samples.

As shown in FIG. 23, with all the tested urine samples of the two felines with cancer, positive C.I. values were obtained. This reveals that the nematodes were attracted to the urine of the felines with cancer. It is apparent from this result that the nematodes are attracted to the urine regardless of the type of the cancer. From the above result, it was confirmed that, with the present invention, it is possible to detect cancer in feline.

### Example 11

### Test for Evaluating Response of Nematodes to Urine of Plural Felines Having Never Developed Cancer

A plate B3 was produced that was identical to the plate of Example 6 in terms of the size and shape of the plate and the shapes of the fall-in cavities, except that the capacity (volume) of a solid medium of the plate B3 was three tenths of that of the plate of Example 6. Left fall-in cavities 2a were filled with a test solution, whereas right fall-in cavities 2b were filled with a physiological saline solution as a control. The test solution used in Example 11 was urine obtained from three felines having never developed cancer. Next, approximately 150 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

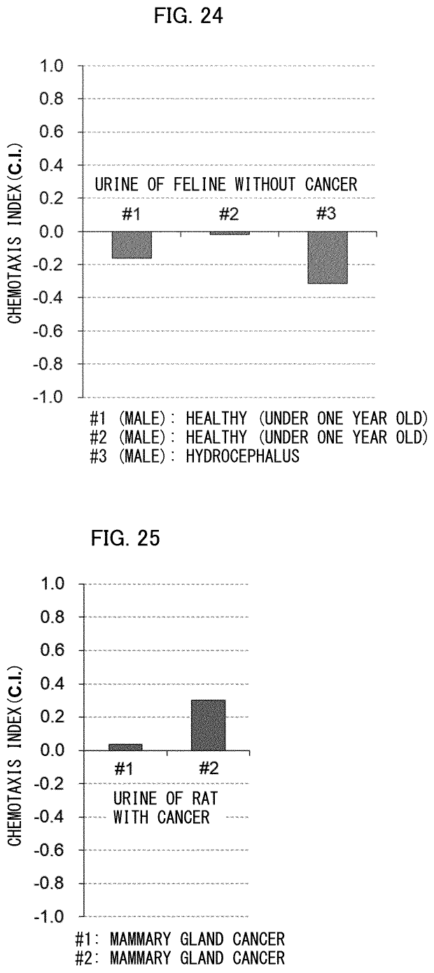

The chemotaxis index (C.I.) was obtained in a similar manner to Example 1, and the response of the nematodes was evaluated. A positive value means attraction to the left fall-in cavities 2a, that is, to the urine of the feline having never developed cancer, whereas a negative value means escaping from the fall-in cavities 2a. Note that the tests on the urine samples of the three felines having never been developed cancer were conducted independently. The three felines included two healthy individuals under one year old and one individual with hydrocephalus. FIG. 24 shows mean values of C.I. obtained in three times of tests conducted independently with use of these urine samples.

As shown in FIG. 24, with all the tested urine samples of the three felines having never developed cancer, negative C.I. values were obtained. This reveals that the nematodes avoided the urine of the felines having never developed cancer. From the above result, it was confirmed that, with the present invention, not only a healthy feline having never developed cancer but also a feline having a disorder other than cancer would be rarely erroneously determined as a feline with cancer.

### Example 12

### Test for Evaluating Response of Nematodes to Urine of Plural Rats with Cancer

A plate B4 was produced that was identical to the plate B2 of Example 3 in terms of the size and shape of the plate and the shapes of the fall-in cavities, except that the capacity (volume) of a solid medium of the plate B4 was three tenths of that of the plate B2. A left fall-in cavity 2a was filled with a test solution, whereas a right fall-in cavity 2b was filled with a physiological saline solution as a control. The test solution used in Example 12 was urine obtained from two rats with mammary gland cancer. Next, approximately 100 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

The chemotaxis index (C.I.) was obtained in a similar manner to Example 3 and the response of the nematodes was evaluated. A positive value means attraction to the left fall-in cavity 2a, that is, to the urine of the rat with cancer, whereas a negative value means escaping from the fall-in cavity 2a. Note that the tests on the urine samples of the two rats with mammary gland cancer were conducted independently. FIG. 25 shows mean values of C.I. obtained in three times of tests conducted independently with use of these urine samples.

As shown in FIG. 25, with all the tested urine samples of the two rats with mammary gland cancer, positive C.I. values were obtained. This reveals that the nematodes were attracted to the urine of the rats with mammary gland cancer. From the above result, it was confirmed that, with the present invention, it is possible to detect cancer in rat.

### Example 13

### Test for Evaluating Response of Nematodes to Urine of Plural Rats Having Never Developed Cancer

A plate B4 identical to that produced in Example 12 was produced. A left fall-in cavity 2a was filled with a test solution, whereas a right fall-in cavity 2b was filled with a physiological saline solution as a control. The test solution used in Example 13 was urine obtained from two rats having never developed cancer. Next, approximately 100 nematodes having been washed in advance with a buffer solution were released onto the center of the plate. The plate was covered with a cover and sealed with tape. Then, the plate was left at least for an hour under a light-shielded environment. Thereafter, images of the fall-in cavities 2a and 2b were captured under a microscope, and the numbers of nematodes caught in the fall-in cavities 2a and 2b were counted based on the images.

(Result)

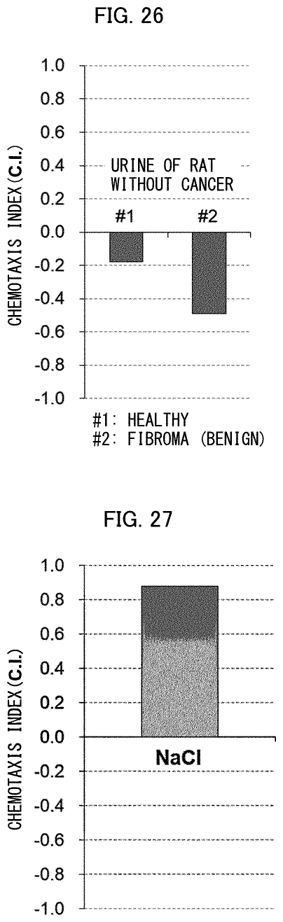

The chemotaxis index (C.I.) was obtained in a similar manner to Example 3, and the response of the nematodes was evaluated. A positive value means attraction to the left fall-in cavity 2a, that is, to the urine of the rat having never developed cancer, whereas a negative value means escaping from the fall-in cavity 2a. The two rats included one healthy individual and one individual with a benign fibroma. FIG. 26 shows mean values of C.I. obtained in three times of tests conducted independently with use of these urine samples.

As shown in FIG. 26, with all the tested urine samples of the two rats having never developed cancer, negative C.I. values were obtained. This reveals that the nematodes avoided the urine of the rats having never developed cancer. From the above result, it was confirmed that, with the present invention, it is possible to discriminate a rat having never developed cancer from a rat with cancer.

### Example 14

### Test for Evaluating Response of Nematodes to Water-Soluble Substance

A test for evaluating a response of nematodes to a water-soluble substance was conducted with use of sodium chloride (NaCl), which is a kind of water-soluble substance. First, a plug containing a test subject at a high concentration was left at one position in a left part of a solid medium, which was a base material of a nematode trap plate B4. Consequently, the test subject was diffused into the solid medium from the plug, so that a concentration gradient was formed. After the plug was left overnight, the plug was removed from the solid medium. Then, a part of the solid medium was hollowed out at the position where the plug had been left, so that a fall-in cavity 2a was formed. Furthermore, a part of the solid medium was further hollowed out at one control position in a right side of the solid medium, so that a fall-in cavity 2b was formed. As a result, the plate B4 was completed. The plug used to form the concentration gradient of NaCl was prepared by hollowing out a cylindrical piece having a diameter of 5 mm from another solid medium that was identical in composition to the solid medium of the nematode trap plate, except that said another solid medium contained NaCl at a final molarity of 100 mM.

The fall-in cavities 2a and 2b were filled with ultrapure water. Next, approximately 100 nematodes having been washed in advance with a buffer solution were released onto the center of the plate, and the plate was covered with a cover and sealed with tape. Then, the plate was left for an hour or longer under a light-shielded environment. Thereafter, images of the fall-in cavities 2a and 2b were captured under a microscope. Based on the images, the numbers of nematodes caught in the fall-in cavities 2a and 2b were counted.

(Result)

Assuming that the number of nematodes caught in the left fall-in cavity 2a, which was formed at the center of the concentration gradient of NaCl, was N₁ and the number of nematodes caught in the right fall-in cavity 2b, which was formed at the control position, was N₂, the value obtained according to the following formula was defined as a chemotaxis index (C.I.). Then, the response was evaluated.

Chemotaxis index (C.I.)=(N₁−N₂)/(N₁+N₂).

A positive value means attraction to the left fall-in cavity 2a, that is, to NaCl, whereas a negative value means escaping from the fall-in cavity 2a. FIG. 27 shows mean values of C.I. obtained in three times of tests conducted independently.

As shown in FIG. 27, positive C.I. values were obtained. This shows that the nematodes were attracted to NaCl. This result exhibits a similar tendency to that of the result of the gustation test of the nematodes conducted with 100 mM NaCl according to the conventional chemotaxis evaluation method. From the above result, it was confirmed that, with the present invention, it is possible to evaluate a response of nematodes to a water-soluble substance.

### Example 15

### Test 3 for Evaluating Response of Soil Nematodes Other than C. elegans to Volatile Substance

Example 15 was conducted for the purpose of studying applicability of the plate of the present invention to evaluation of a response of nematodes other than C. elegans. Specifically, the test was conducted to examine a response of a hetero group M of free-living soil nematodes (collected at Ogasawara village, Tokyo, Japan) and a hetero group H of free-living soil nematodes (collected at Takayama city, Gifu, Japan) with respect to a volatile substance. A plate A1 identical to that produced in Example 1 was produced. Left fall-in cavities 2a were filled with a test solution, whereas right fall-in cavities 2b were filled with a buffer solution as a control. The test solution used in Example 15 was a diluted solution (1 mM) of vanillin, which is a kind of volatile substance. Next, approximately 150 nematodes having been washed in advance with the buffer solution were released onto the center of the plate, and the plate was covered with a cover and sealed with tape. Then, the plate was left at least for an hour under a light-shielded environment. Thereafter, images of the fall-in cavities 2a and 2b were captured under a microscope. Based on the images, the numbers of nematodes caught in the fall-in cavities 2a and 2b were counted.

(Result)

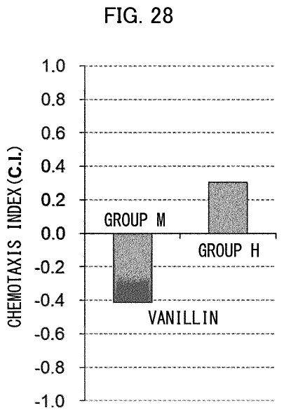

The chemotaxis index (C.I.) was obtained in a similar manner to Example 1, and the response of the nematodes was evaluated. A positive value means attraction to the left fall-in cavities 2a, that is, to vanillin, whereas a negative value means escaping from the fall-in cavities 2a. Note that the tests involving use of the nematode group M and the nematode group H were conducted independently. FIG. 28 shows the test result.

As shown in FIG. 28, for the group M, which was the group of free-living soil nematodes collected at Ogasawara village, Tokyo, Japan, a negative C.I. value was obtained. This reveals that the nematodes avoided vanillin, which is a kind of food constituent. Meanwhile, for the group H, which was the group of free-living soil nematode collected at Takayama city, Gifu, Japan, a positive C.I. value was obtained. This shows that the nematodes were attracted to vanillin. From these results, it was found that the free-living soil nematodes derived from different sites have different preferences. The above results support the fact that, with the present invention, it is possible to carry out screening for electing, from among a hetero group of nematodes, only nematodes suitable for a certain environment on the basis of the preference of the nematodes.

It is apparent from the results of the above-described Examples that, with the present invention, it is possible to conduct a test for evaluating a response of nematodes to a test solution, regardless of the size of the nematode trap plate, the number of fall-in cavities to be formed and the sizes thereof (i.e., the capacity (volume) of the test solution), the kind of chemical substance solution to be used as the test solution and the kind of biological sample to be used, the kind of liquid to be used for dilution of the test solution, the kind of liquid to be poured into the control fall-in cavity, the kind of nematodes, and the like. Particularly, it is apparent that, in a case where undiluted urine of mammal or a diluted solution thereof is used as the test solution, it is possible to determine whether or not the testee (animal) has cancer on the basis of the result of the chemotaxis test of the nematodes carried out in accordance with the present invention, regardless of the species, the sex, the age, the presence or absence of cancer, the type of cancer, and the like of the testee.

### Example 16

### Generation of Calibration Data used to Obtain the Number of Nematode Individuals in Accordance with Method (B1) Involving use of Shading of Group of Nematodes

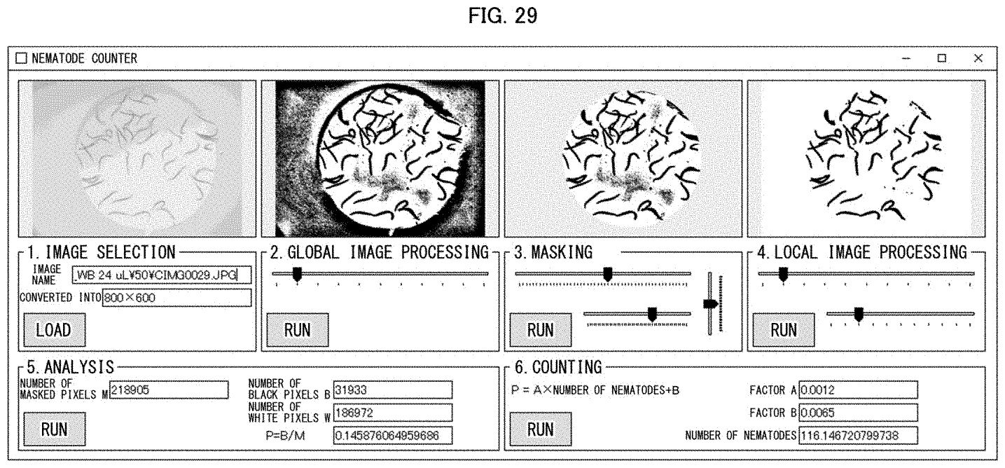

A plate B5 having a single fall-in cavity 2a formed in a solid medium was produced. The fall-in cavity 2a was filled with a buffer solution. Next, nematodes that were of the same kind as that used in Example 1 and that had been washed in advance with the buffer solution were introduced into the fall-in cavity 2a while the number of nematodes to be supplied was increased in several steps in this order: five, ten, 20, 30, 40, and 50. Three bright-field still images of the fall-in cavity were captured with a digital camera mounted on a microscope at each step. The area of an image corresponding to the nematode individuals in the fall-in cavity in the still image, that is, the number of black pixels observed after binarization was calculated by a self-made image analyzing program, installed in a general-purpose computer, for automatically counting the number of nematode individuals. FIG. 29 shows one example of a processing screen of the image analyzing program.

(Result)

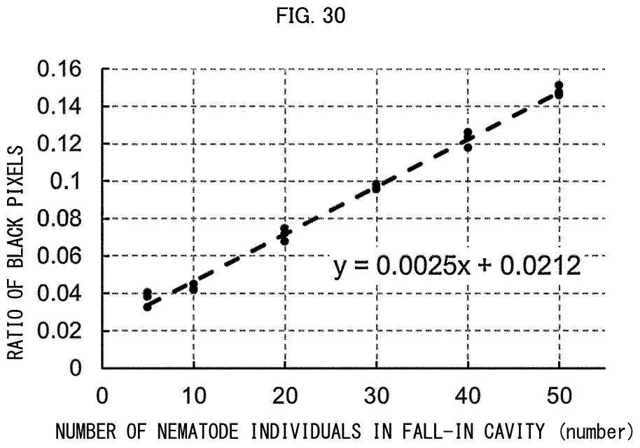

Plotted along the horizontal axis are the numbers of nematode individuals introduced into the fall-in cavity 2a. Meanwhile, plotted along the vertical axis are the ratios, derived through image analysis, of the black pixels corresponding to the nematode individuals with respect to the entire image. FIG. 30 shows the result. The bold broken line is an approximation curve of the values obtained from the three images captured for each number of individuals, and is a standard curve used in calibration.

It is apparent from FIG. 30 that the number of nematode individuals in the fall-in cavity is proportional to the area of the image corresponding to the nematodes and thus the number of nematodes caught in the fall-in cavity can be estimated from the still image of the fall-in cavity.

### INDUSTRIAL APPLICABILITY

The present invention is applicable to various industrial fields such as medical science and medical service, life science, veterinary science and veterinary service, agriculture and forestry, food field, quantum science, and environmental field.

### REFERENCE SIGNS LIST

1: Nematode trap plate

2a to 2d: Fall-in cavity (recess)

3: Cover

4a, 4b: Through hole (hole)

5: Bottom surface

6a, 6b: Tube (tubular member)

10, 10a: Container

11: Solid medium (solid phase)

20a to 20c: Hollowing-out tool

21: Grip

22a, 22b: Tubular part

23: Needle

30: Main body part (main body)

31: Plate-shaped member

32: Insertion hole

40a to 40d: Protrusion

41a to 41d: Protrusion main body

42, 45: Insertion part

43: Fixing pin

44: Clip part

46: Nonslip cap

50: Forming mold

51: Main body outer frame (main body)

52: Fit-in part

53: Protrusion

1. A nematode trap plate comprising:
a container; and
a solid phase formed in the container,
the solid phase allowing nematodes to move over a surface of the solid phase,
the surface of the solid phase having at least one recess for catching one or some of the nematodes.

2. The nematode trap plate as set forth in claim 1, wherein
the container has a bottom surface having a transmittance of not less than 70% for light having a wavelength of 360 nm to 1500 nm.

3. A forming mold for producing the nematode trap plate recited in claim 1, comprising:
a main body; and
at least one protrusion provided in the main body, said at least one protrusion corresponding to said at least one recess,
the forming mold being fixable to the container.

4. The forming mold as set forth in claim 3, wherein
said at least one protrusion is detachably attachable to the main body.

5. A container using for the nematode trap plate recited in claim 1, comprising:
a hole provided at a part of a bottom surface of the container at which part said at least one recess is to be formed, the hole being designed to receive a tubular member that is to be inserted into the hole to form said at least one recess; and
a space below at least the part of the bottom surface at which part the hole is provided.

6. A method for producing the nematode trap plate recited in claim 1, comprising the step of:
forming said at least one recess in the solid phase, wherein
in the step of forming said at least one recess, said at least one recess is formed by
(a) forming the solid phase with a forming mold being fixed to the container, the forming mold being for producing the nematode trap plate, the forming mold including a main body and at least one protrusion provided in the main body, said at least one protrusion corresponding to said at least one recess, the forming mold being fixable to the container, or
(b) forming the solid phase on the container, followed by hollowing out a part of the solid phase.

7. A method for producing a nematode trap plate recited in claim 1, comprising the step of:
forming said at least one recess in the solid phase, wherein
in the step of forming said at least one recess, said at least one recess is formed by
(a) inserting, into the hole of the container recited in claim 5, a tubular member that has a bottom at a lower end of the tubular member and that is opened at an upper end of the tubular member, so that the tubular member is fixed at the hole,
(b) forming the solid phase so that the surface of the solid phase is lower in height than the upper end of the tubular member, and
(c) after forming the solid phase, pushing down the tubular member until the upper end of the tubular member coincides in height with the surface of the solid phase.

8. A method for evaluating a response of nematodes to a test subject, comprising the steps of:
preparing a test plate including the nematode trap plate recited in claim 1 in which a test subject has been supplied into said at least one recess or to an area around said at least one recess;
supplying nematodes to a certain position on the surface of the solid phase; and
after a certain duration has elapsed, obtaining the number of nematodes caught in said at least one recess or a factor that correlates with the number of nematodes caught in said at least one recess, wherein
said at least one recess is filled with a liquid.

9. The method as set forth in claim 8, wherein
the test subject encompasses a body fluid of mammal.

10. The method as set forth in claim 8, wherein
the test subject encompasses urine of human, canine, feline, monkey, mouse, rat, or marmot.

11. A method for evaluating a response of nematodes to a temperature, comprising the steps of:
adjusting a temperature inside said at least one recess or a temperature of an area around said at least one recess in the nematode trap plate recited in claim 1 so that the temperature is at a desired value;
supplying nematodes to a certain position on the surface of the solid phase; and
after a certain duration has elapsed, obtaining the number of nematodes caught in said at least one recess or a factor that correlates with the number of nematodes caught in said at least one recess, wherein
said at least one recess is filled with a liquid.

12. The method as set forth in claim 8, wherein
after the certain duration has elapsed, an image of said at least one recess is captured and image processing is carried out on the image thus captured so as to obtain the number of nematodes caught in said at least one recess or the factor that correlates with the number of nematodes caught in said at least one recess.

13. The method as set forth in claim 8, wherein
the nematodes to be used are nematodes having a fluorescent probe incorporated therein, and
the number of nematodes caught in said at least one recess is calculated from a total of fluorescence intensities of the nematodes caught in said at least one recess.

14. A method for evaluating behaviors of nematodes, comprising the steps of:
supplying nematodes to a certain position on the surface of the solid phase of the nematode trap plate recited in claim 1; and
after a certain duration has elapsed, obtaining the number of nematodes caught in said at least one recess or a factor that correlates with the number of nematodes caught in said at least one recess, wherein
said at least one recess is filled with a liquid.

15. A nematode trap test kit comprising
the nematode trap plate recited in claim 1; and
a cover for maintaining an environment on the solid phase so as to be fixed, the cover having a transmittance of not less than 70% for light having a wavelength of 360 nm to 1500 nm.

16. The nematode trap test kit as set forth in claim 15, further comprising nematodes to be used in a test.

17. A method for screening cancer, comprising the steps of:
preparing a test plate including the nematode trap plate recited in claim 1 in which urine obtained from a testee has been supplied into said at least one recess or to an area around said at least one recess;
supplying nematodes to a certain position on the surface of the solid phase; and
after a certain duration has elapsed, obtaining the number of nematodes caught in said at least one recess or a factor that correlates with the number of nematodes caught in said at least one recess, wherein
said at least one recess is filled with a liquid, and
a possibility that the testee has cancer is determined based on the number of nematodes caught in said at least one recess.

18. The method as set forth in claim 17, wherein
the testee is human.

19. The method as set forth in claim 17, wherein
the testee is canine or feline.

20. The method as set forth in claim 17, wherein
the testee is monkey, mouse, rat, or marmot
